(19) United States
(12) Patent Application Publication
(10) Pub. No.: US 2016/0299149 A1
(43) Pub. Date: Oct. 13, 2016

(54) ASSAYS AND METHODS FOR CELL PROLIFERATION-TARGETED TREATMENT THERAPIES

(71) Applicants: Otto Phanstiel, IV, Oviedo, FL (US); Deborah Altomare, Oviedo, FL (US); Laurence von Kalm, Orlando, FL (US)

(72) Inventors: Otto Phanstiel, IV, Oviedo, FL (US); Deborah Altomare, Oviedo, FL (US); Laurence von Kalm, Orlando, FL (US)

(73) Assignee: University of Central Florida Research Foundation, Inc., Orlando, FL (US)

(21) Appl. No.: 15/074,993

(22) Filed: Mar. 18, 2016

Related U.S. Application Data
(60) Provisional application No. 62/134,895, filed on Mar. 18, 2015.

(51) Int. Cl. G01N 33/574; A61K 31/198; A61K 31/404; C12Q 1/68; A61K 31/137

(52) U.S. Cl. CPC G01N 33/57496; G01N 33/57492; C12Q 1/6886; C12Y 306/03; A61K 31/137; A61K 31/404; A61K 31/198; C12Q 2600/16; C12Q 2600/106; C12Q 2600/158; G01N 2333/914; G01N 2333/4703; G01N 2333/47

(57) ABSTRACT

Assays are described which measure polyamine transport activity and markers associated with polyamine transport and metabolism. These data are then used for the selection of treatments using therapies which target polyamine transport and polyamine metabolism. The assay includes a substrate to which a protein-containing cell sample can bind, a solution comprising a first antibody specific against ATP13A3, wherein the first antibody is configured to bind to ATP13A3 protein on the substrate, a solution comprising a second antibody specific against the first antibody, the second antibody comprising an enzyme linked thereto, wherein the second antibody is configured to bind to the first antibody. The assay further includes a substrate specific to the enzyme, wherein upon combining the substrate and the enzyme, the amount of enzyme in the solution can be identified, wherein said amount of enzyme identified is proportional to the amount of ATP13A3 protein in the sample, wherein said ATP13A3 protein is indicative of polyamine transport in the cells of the sample. In addition, primary antibodies covalently attached to respective fluorophores can be used to directly measure the relative expression levels of these biomarkers in histological samples. In addition, several biomarkers are described which allow for therapy selection based upon the expression and relative ratios of specific proteins associated with polyamine transport (c-myc, ATP13A3 Cav-2, and Cav-1 as well as c-Raf).

### BACKGROUND

The polyamines (putrescine 1, spermidine 2a and spermine 3, FIG. 1) are ubiquitous low-molecular-weight aliphatic amines bearing multiple amine groups¹. At physiological pH, polyamines exist as tethered polycations, and function via their interactions with anionic biomolecules (e.g., proteoglycans, DNA and RNA)². Polyamines are essential growth factors which play critical roles in cell proliferation, including their involvement in transcription, translation and regulation of chromatin structure as well as in the control of apoptosis and cytoskeletal dynamics¹. Moreover, spermidine 2a provides the sole source of the aminobutyl fragment necessary for generation of hypusine, a unique amino acid critical for the function of the eukaryotic initiation factor-5A¹. In this regard, polyamines are essential for life and their intracellular levels are tightly controlled by mechanisms which allow rapid fluctuations to respond to specific needs.

In contrast to inorganic cations, polyamines can be biosynthesized and catabolized. Intracellular levels of polyamines are maintained by a subtle equilibrium between a complex enzymatic machinery (controlling biosynthesis and catabolism), and systems that allow import and export¹. Although the molecular biology of polyamine metabolism in mammalian cells is extensively known, polyamine transport remains an observable, yet elusive phenomenon. While polyamine transporters have been characterized in the bacteria and unicellular eukaryotes, the identities of the genes involved in the polyamine transport system (PTS) in any multicellular organism were, until recently, unknown⁽²⁻¹³⁾.

Even with recent advances, in most respects the mammalian PTS is still a black box. Mammalian cells import polyamines by carrier-mediated, energy- and Natdependent mechanisms⁽¹⁵⁻¹⁷⁾. Observations in many cell-types suggest that transport may be more complex than a single transporter. Transporters with different affinities for putrescine, spermidine and spermine have been biochemically characterized⁽¹⁸⁻²⁵⁾, and differences in Na⁺-dependence for import have been reported⁽²⁰⁻²³⁾.

The PTS is fully integrated into the cellular regulatory system for the control of intracellular polyamine levels^{(19, 24-25)}.

Many cancer cell types have high polyamine biosynthesis and high polyamine transport activity, presumably to sustain their rapid growth rates. While polyamine biosynthesis inhibitors like difluoromethylornithine (DFMO) are known and have a mixed clinical history in the treatment of cancers²⁶, there is no commercially-available polyamine transport inhibitor (PTI)⁽²⁷⁻²⁹⁾.

These transport inhibitors are needed because cancers can circumvent DFMO therapy by importing exogenous polyamines²⁴. Indeed, there is an opportunity to uncover new biomarkers which can help clinicians identify tumors which will best respond to DFMO therapy or combination therapies involving DFMO and a PTI. Unfortunately, both PTI-design and biomarker development are hampered without molecular knowledge of the PTS itself.

There are at least two models that describe polyamine entry into cells, either of which may be operative depending on cell type and/or the molecular structure of the polyamine itself (FIG. 2). In one model, Poulin et al provided evidence in favor of a classical transporter, potentially similar to the ABC transporters in unicellular organisms, and requiring an electronegative membrane potential to transport polyamines across the membrane (Model A in FIG. 2)^{(3, 30)}. In a second model, the positively-charged polyamine interacts with a negatively-charged structure present on the external side of the cell membrane (FIG. 2; Model B). Heparan sulfate (HS) on the proteoglycan (PG) core protein, glypican-1, has been proposed by Belting et. al. as the putative polyamine-binding site⁽³¹⁻³³⁾.

The membrane-bound polyamine is then imported into cells via caveolin dependent endocytosis⁽³⁵⁻³⁶⁾.

A similar model invoking receptor-mediated endocytosis as a route for polyamine entry is also supported by the initial work of Poulin, Soulet, et al., who used polyamines conjugated to fluorescent probes to explore polyamine transport and polyamine intracellular trafficking.

Belting observed dramatic reduction of spermine uptake in proteoglycan-deficient CHO cell mutants, which suggested that proteoglycans are necessary for polyamine transport. In support of Model B, Belting demonstrated a high-affinity binding site for spermine on HS and showed that cells with an inability to process HS (via nitric oxide) had significantly reduced spermine uptake. However, there is contradictory evidence which showed that polyamine transport was not significantly altered (20% reduction) in CHO cells defective in HS biosynthesis⁽³¹⁻³²⁾. Intriguingly, these HS-deficient cells had up-regulated chondroitin sulfate (CS) suggesting that a different carbohydrate could also mediate uptake⁽³¹⁻³²⁾. These observations suggest that cells can alter their cell-surface glycan composition to bind and import polyamines from outside the cell. Due to the fact that neither spermidine nor putrescine were evaluated in the prior art, further studies would be required to delineate the nature of the transport receptor and if the other polyamines share the same uptake mechanism.

### BRIEF DESCRIPTION OF THE DRAWINGS

The following drawings form part of the present specification and are included to further demonstrate certain embodiments of the present invention. The invention may be better understood by reference to one or more of these drawings in combination with the detailed description of specific embodiments presented herein.

FIG. 1 provides native polyamine structures 1-3, examples, wherein the similarity in molecular structure between homospermidine and spermidine can be seen.

FIG. 2 is a diagram of Polyamine Biosynthesis and Transport pathways. ODC (ornithine decarboxylase) is a key polyamine biosynthetic enzyme and is naturally regulated by Antizyme and can be inhibited by the drug DFMO. Model A (Poulin et al involves a membrane bound transporter and formation of polyamine sequestering vesicles (PSVs)). Model B (Belting, et al) involves heparan sulfate proteoglycans and glypican as cell surface recognition elements for polyamine capture followed by caveolin-dependent endocytosis. The models shown may be mutually inclusive or both operative.

FIG. 3 is a diagram of an embodiment of a Polyamine Oncologic Loop showing the cross-talk between several cell signaling pathways and the fact that polyamines which are often upregulated by these oncogenic pathways can further activate specific targets in these pathways.

FIG. 4 is a diagram showing an Ant44 assay, where a PTI will inhibit the uptake of a cytotoxic fluorescent polyamine probe Ant44 into a cell.

FIG. 5 demonstrates CHO cells are protected from a cytotoxic dose of the polyamine transport ligand Ant44 (3 μM) in the presence of a polyamine transport inhibitor (PTI) GW5074. As shown in the graph CHO cells dosed with Ant44 (3 μM) gave 38% viability. Cell viability was increased in a dose dependent manner when the Ant44 concentration was held fixed at 3 μM and the GW5074 PTI concentration was increased from 0 to 10 μM (as shown in the x-axis). The GW5074 PTI compound was not toxic to CHO cells at 10 μM. A similar rescue from Ant44 was noted for the addition of exogenous spermidine and suggests that the Ant44 compound uses the polyamine transport system for cell entry.

FIG. 6A is a diagram showing an assay wherein the uptake of rescuing dose of spermidine into DFMO-treated cells is blocked by a PTI (GW 5074).

FIG. 6B provides the chemical structures of validated PTIs. Compounds 6a and 6b are polyamine based PTIs and GW5074 is a non-polyamine based PTI which is also a c-Raf inhibitor.

FIG. 7A-B provide a table and a graph showing a combination therapy of DFMO+PTI (GW5074) in the presence and absence of exogenous spermidine.

FIG. 8 provides an illustration and a table showing different cell line sensitivity to DFMO and spermidine rescue challenge. The rescue challenge experiment doses cells with the respective IC₅₀ dose of DFMO for that cell line and measures the viability of cells with and without added spermidine (1 μM). Cell lines without added spermidine give the expected 50% viability due to the presence of DFMO. Cell lines with added spermidine with highly active polyamine transport systems import the exogenous spermidine and rescue themselves from the IC50 dose of DFMO and give significantly higher viability values than 50% viability. These are highlighted in red. Cell lines with low polyamine transport activity are not rescued to a significant extent even though a rescuing dose of spermidine is available and give viabilities near 50%.

FIGS. 9A-B provide graphical illustrations of the modulation of ATP13A3 and Cav-1, respectively, by DFMO and DFMO+Spermidine in L3.6pl human pancreatic cancer cells after 72 h incubation at 37° C. In FIG. 9A ATP13A3 protein levels after 72 h incubation at 37° C. are shown in L3.6pl (top) and Bx-PC3 (bottom) pancreatic cancer cells in the presence of DFMO or DFMO+Spd as analyzed by Western blot. Expression was normalized using β-actin levels. Both cell lines showed a significant increase in relative ATP13A3 expression in the presence of the 48 h IC₅₀ DFMO dose (e.g., 8 mM) or DFMO+(1 μM Spermidine). In FIG. 9B (top and bottom), the levels of Cav-1 after 72 h incubation at 37° C. were not dramatically changed in either L3.6pl or BxPC-3 pancreatic cancer cells in the presence of DFMO indicating that the basal Cav-1 levels do not change dramatically with DFMO addition. Note an increase in Cav-1 levels would stabilize caveolae at the cell surface and reduce the uptake of polyamines via a caveolin dependent pathway. A decrease in Cav-1 levels would increase polyamine import.

FIG. 10 is a table providing the Relative Protein expression of Cav-1, ATP13A3 and c-myc in human pancreatic cancer cell lines along with the respective Vₘₐₓ values for ³H spermidine (Spd) import and the relative affinity Kₘ values determined for spermidine. Entries represent 100%×protein expression/beta actin expression as shown in FIG. 10 along with the Vₘₐₓ value already provided. The Western blots were quantified by Image J software (NIH) and normalized by dividing by the beta-actin expression level for each cell line. Entries represent protein expression and are expressed in relative %, with the L3.6pl cell expression levels set to 100%. Cells with high basal ATP13A3 and c-myc levels (e.g., L3.6pl cells) gave high rates of polyamine import (high Vmax) and lower binding affinity (higher Kₘ values) suggesting that a lower affinity transport system is operative in cell lines with high polyamine transport activity.

FIG. 11A provides a western blot analysis of proteins in the presence of DFMO, spd, GW5074 PTI and varying combinations thereof as shown. The most significant changes were noted for c-myc and ATP13A3. FIG. 11B provides a graphical illustration of modulation of c-Myc and ATP13A3 protein expression in human L3.6pl cells (normalized vs beta-actin control) in the presence of a PTI (GW5074), DFMO and Spermidine.

FIG. 12A-B provides a graphical illustration and a table with results demonstrating the ability of GW5074 and the trimer44NMe (‘PTI’ in the figure) to inhibit in vivo pancreatic tumor growth in combination with DFMO. FIG. 13B is a table representing results of human L3.6pl Pancreatic Tumor in Nude Mice-Combination with 1% DFMO. Note PTI refers to the polyamine based PTI, trimer44NMe.

FIGS. 13A-B include a graphical illustration and a table showing Pan02 in vivo pancreatic tumor growth was inhibited with the trimer44NMe PTI and/or GW5074 in combination with DFMO. FIG. 13B is a table providing results of Mouse Pan02 Pancreatic Tumor in C57Bl/6 Mice-Combination with 1% DFMO and GW5074 or PTI, or a combination thereof. Note: TTI′ denotes the polyamine based trimer 44NMe PTI agent.

FIG. 14 illustrates histological stained images of Ki67, a proliferation marker, and cleaved caspase in the presence of DFMO or DFMO+ the trimer44NMe PTI

FIG. 15 shows a signaling pathway for melanoma BRAF and polyamine transport targeting compounds, e.g., compound A to inhibit polyamine transport in the signaling pathway. Compound A, for example, may inhibit polyamine import into cells via the polyamine transport system (PTS) by, for example outcompeting native polyamines for cell entry. Compound A may also inhibit proliferation of polyamines as provided in the diagram.

FIG. 16 provides molecular structure for polyamine transport selective probes and targeting compounds, Ant44, compound A, and compound B.

FIG. 17A shows a Western blot analysis of Caveolin proteins in melanoma cell lines showing high levels of Cav-1 and low levels of Cav-2 in the most resistant melanoma cell line (LOX IMVI) to compound A. See Eustace, A. J.; Kennedy, S.; Larkin, A. M.; Mahgoub, T.; Tryfonopoulos, D.; O'Driscoll, L.; Clynes, M.; Crown, J.; O'Donovan, N. Predictive biomarkers for dasatinib treatment in melanoma. Oncoscience 2014, 1 (2), 158-166.

FIG. 17B shows selective targeting of melanomas of various cell types and cell sensitivity to compound A (10 μM) is shown in the NCI 60-cancer cell screen. Graphical bars extending to the right of the 0 growth percent line indicates growth inhibition by compound A of those specific cell lines. MALME-3M was the most sensitive cell line to compound A as shown, which includes low Cav-1 and high Cav-2 levels. Compound A gave a 96 h IC₅₀ of 14 nM in MALME-3M.

FIG. 17C shows a graphical illustration of selective targeting of melanomas and cell sensitivity to anthracenyl derivative compound B (10 μM) in the NCI 60-cancer cell screen. As in FIG. 18B, a graphical bar extending to the right of the 0 growth (central line) in FIG. 18C indicates growth inhibition by compound B. MALME-3M (low Cav-1 and high Cav-2) was the most sensitive cell line to compound B. Compound B gave a 96 h IC₅₀ of 62 nM in MALME-3M and was used in confocal microscopy studies showing rapid cell uptake and nuclear import in cultured MALME-3M cells (see FIG. 21).

FIG. 18 provides a graphical illustration of cells incubated for 96 h at 37 degrees Celsius with compound A at 0.02 μM (MALME-3M cells) and 0.8 μM (MALME-3 control cells) demonstrating the percent viability of the cells. Aminoguanidine (AG) at 1 mM was determined to be non-toxic and was incubated with cells for 24 h prior to compound addition. Control represents the untreated cells. All experiments were performed in triplicate. Spermidine (Spd, 100 μM) was non-toxic to both cell lines. As provided in the figure, compound A competes with Spd for cell entry. Compound A is a valid polyamine transport probe.

FIG. 19A provides a graphical illustration of ATP13A3 protein levels in L3.6pl cells as analyzed by Western blot. Expression was normalized by dividing by β-actin protein levels. Decreased ATP13A3 protein expression was observed in the presence of the ATP13A3 siRNA vs the scrambled siRNA (p<0.05; each at 75 nM).

FIG. 19B provides a graphical illustration of the effect of scrambled siRNA vs. ATP13A3 siRNA on relative L3.6pl cell viability % when challenged with a 48 h IC50 dose of DFMO (8 mM) and a rescuing dose of Spd (1 μM). The data provided shows reduced ATP13A3 expression and associated reduced rescue by Spd (p, 0.01) by the ATP13A3 siRNA. This demonstrates the involvement of ATP13A3 in polyamine transport.

FIG. 20 is a confocal microscopy view of MALME-3M cells incubated with the PTS-selective compound B (2.5 μM). Briefly, the MALME-3M cells were mounted on microscope slides and were placed into a Tokai Hit Chamber to control humidity, temperature and CO₂ levels over the course of the experiment. A NIKON A1 Laser scanning confocal microscope equipped with a 405 nm excitation laser light and a transmitted light detector was used to acquire images over time. The image was acquired at 4 h 51 min after addition of B. The time-course study showed a dose dependent uptake of compound B over time. B, which appears blue under the 405 nm laser excitation, becomes very apparent at the 5 hour time point inside the nuclear compartment. This demonstrates cellular uptake of this molecule and a time-course preferred localization to the nucleus.

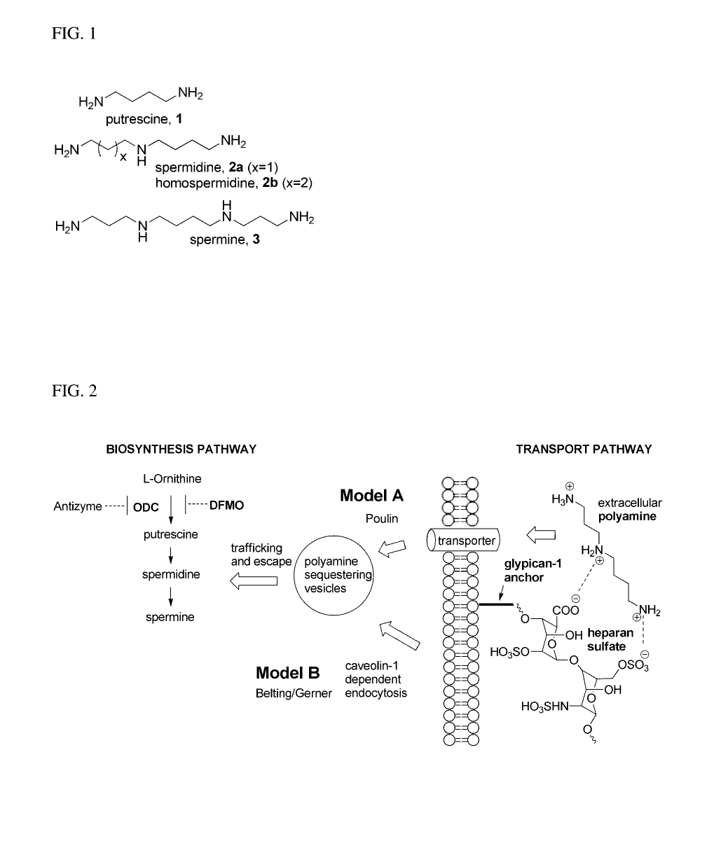

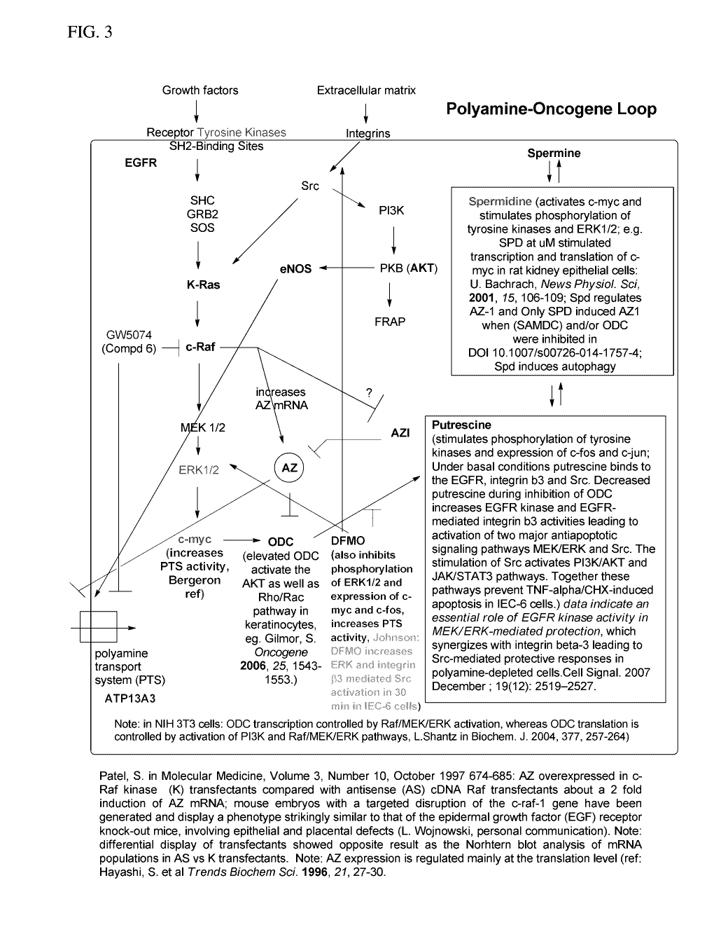

### DEFINITIONS

The term “therapeutically effective amount” as used herein means an amount that achieves the intended therapeutic effect of terminating targeted cells treated with a drug, or reducing the number of the cells targeted by the drug in a subject. The full therapeutic effect does not necessarily occur by administration of one dose and may occur only after administration of a series of doses. Thus, a therapeutically effective amount may be administered in one or more administrations per day for successive days. A series of doses administered over the course of a period of time is referred to herein as a “regimen.”

The term “preventing” a disease as used herein refers to reducing or delaying the onset of the disease in a subject who is at high risk of developing the disease. In a non-limiting embodiment, the disease may include colorectal cancer, and in a further non-limiting embodiment, the disease may include inflammatory bowel diseases (IBD) like Crohn's disease and ulcerative colitis.

The term “treating” refers to a disorder or disease described herein including but not limited to cancer or other proliferative disorders. In a non-limiting embodiment, the disease may include pancreatic cancer, and in a further non-limiting embodiment, the disease may include a primary pancreatic cancer and/or a metastatic pancreatic cancer.

The term “sample” as used herein refers to a cell-containing sample of a patient, including a tissue, or a tumor, blood, tears, urine or feces sample, in non-limiting embodiments. In some embodiments described herein, the term sample is referred to as a blood sample, but any other type of sample described herein or known to those skilled in the art may be used as a substitute sample, or in addition to a blood sample. The term “cell sample” as used herein may include whole cells, lysed cells, cell homogenate, a cell tissue layer, or any other type of cells ample described herein or known to those skilled in the art.

The terms “animal” “patient” or “subject” as used interchangeably herein refer to any animal for example mammals, including, but not limited to humans, primates, dogs, cattle, cows, horses, kangaroos, pigs, sheep, goats, cats, rabbits, rodents, and transgenic non-human animals, and the like, which are to be the recipient of a particular treatment. Typically, the terms “animal” “subject” and “patient” are used interchangeably herein in reference to a human subject or a rodent. The preferred animal, patient, or subject is a human.

The term “solid support” or “substrate” refers to any material that can be modified to contain discrete individual sites appropriate for the attachment or association of a capture binding ligand. Suitable substrates include metal surfaces such as gold, electrodes, glass and modified or functionalized glass, plastics (including acrylics, polystyrene and copolymers of styrene and other materials, polypropylene, polyethylene, polybutylene, polycarbonate, polyurethanes, Teflon, derivatives thereof, etc.), polysaccharides, nylon or nitrocellulose, resins, mica, silica or silica-based materials including silicon and modified silicon, carbon, metals, inorganic glasses, fiberglass, ceramics, GETEK (a blend of polypropylene oxide and fiberglass) and a variety of other polymers and their functionalized derivatives.

The term “measuring” means methods which include detecting the presence or absence of marker(s) in the sample, quantifying the amount of marker(s) in the sample, and/or qualifying the type of biomarker. Measuring can be accomplished by methods known in the art and those further described herein, including but not limited to microarray analysis (with Significance Analysis of Microarrays (SAM) software), SELDI, quantitative polymerase chain reaction (PCR), quantitative reverse transcription polymerase chain reaction-(RT-PCR), enzyme-linked immunosorbent assay (ELISA) and immunoassays to measure either RNA and/or protein levels in biological samples. Any suitable methods can be used to detect and measure one or more of the markers described herein. These methods include, without limitation, mass spectrometry (e.g., laser desorption/ionization mass spectrometry), fluorescence (e.g. sandwich immunoassay), surface plasmon resonance, ellipsometry, Western blot analysis, ELISA, immunoassays, RT-PCR or PCR and atomic force microscopy.

The term “label” may include a radioactive, a fluorescent, a chemiluminescent, a dye, an enzyme, or a histidine label, or any other suitable label known in the art.

The term “binding molecule(s)” may include antibodies or other binding molecules specific to and configured to bind other antibodies, in a non-limiting embodiment. In other non-limiting embodiments, the binding molecules may bind multiple antibodies. The binding molecules may be provided as attached to or associated with a substrate to which an antibody-bound molecule can bind.

“Detect” refers to identifying the presence, absence or amount of the object to be detected.

The phrase “differentially present” refers to differences in the quantity and/or the frequency of a marker present in a sample taken from subjects having symptoms of cancer as compared to a control subject or a reference subject or sample. For example, some markers described herein are present at an elevated level in samples of subjects compared to samples from control subjects, e.g., c-myc, Cav-2 and ATP13A3. In contrast, other markers described herein are present at a decreased level in samples of cancer subjects compared to samples from control subjects, e.g., Cav-1. Furthermore, a marker can be a polypeptide or a nucleic acid, which is detected at a higher frequency or at a lower frequency in samples of human cancer subjects compared to samples of control subjects.

Furthermore, a marker can be a polypeptide or nucleic acid, which is detected at a higher frequency or at a lower frequency in samples of unaffected tissue from subjects with cancer compared to samples of affected tissue from subjects with cancer (e.g., melanoma).

Furthermore, a marker can be a polypeptide or nucleic acid, which is detected at a higher frequency or at a lower frequency in samples of human unaffected tissue from cancer subjects (e.g., melanoma) compared to samples of control subjects (e.g., without cancer).

Furthermore, a marker can be a polypeptide or nucleic acid, which is detected at a higher frequency or at a lower frequency in samples of human affected tissue from cancer subjects (e.g., melanoma) compared to samples of control subjects (without cancer).

A marker can be differentially present in terms of quantity, frequency or both.

“Affected tissue,” as used herein refers to tissue from a cancer subject that is grossly diseased tissue (e.g., skin (epidermis and/or dermis) lymph nodes, metastatic sites or tumor tissue, e.g., pancreas, brain, lung, bone, liver, skin, or melanocytes).

“Unaffected tissue,” as used herein refers to a tissue from a cancer subject that is from a portion of tissue that does not have gross disease present, for example tissue that is about 1, 2, 5, 10, 20 or more cm from grossly-diseased tissue.

“Normal cells” as used herein refers to cells of unaffected tissue.

“Basal level” as used herein in relation to a level of a protein relates to a level of the protein in a normal cell.

“Relative to normal cells” as used herein to describe a protein level in a cell of affected tissue pertains to relative comparison of the protein level in a cell of unaffected tissue. Typically, the cell of the unaffected tissue is of a similar source to that of the affected tissue. For example, a protein level of cells of affected tissue in the breast is compared to the protein level of cells in unaffected tissue in the breast.

“At least 50-100% higher than basal level” as used herein means that a determined value is at least 50% or at least 100% higher than a basal level, or at least a percentage value higher than basal level of each integer percent within the range of 50-100%. For example, at least 50-100% higher than basal level includes at least 51%, 52%, 60%, 70%, 85% etc. higher than basal level.

As used herein, “array” or “microarray” refers to an array of distinct polynucleotides, oligonucleotides, polypeptides, or oligopeptides synthesized on a substrate, such as paper, nylon, or other type of membrane, filter, chip, glass slide, or any other suitable solid support. Arrays also include a plurality of antibodies immobilized on a support for detecting specific protein products. There are several microarrays that are commercially available. A microarray may include one or more biomarkers disclosed herein. A panel of about 20 biomarkers as nucleic acid fragments can be included in an array. The nucleic acid fragments may include oligonucleotides or amplified partial or complete nucleotide sequences of the biomarkers. The term “consisting essentially of” generally refers to a collection of markers that substantially affects the determination of the disorder and may include other components such as controls.

In an embodiment, the microarray is prepared and used according to the methods described in U.S. Pat. No. 5,837,832, Chee et al.; PCT application WO95/11995, Chee et al.; Lockhart et al., 1996. Nat Biotech., 14:1675-80; and Schena et al., 1996. Proc. Natl. Acad. Sci. 93:10614-619, all of which are herein incorporated by reference to the extent they relate to methods of making a microarray. Arrays can also be produced by the methods described in Brown et al., U.S. Pat. No. 5,807,522. Arrays and microarrays may be referred to as “DNA chips” or “protein chips.”

A variety of clustering methods are available for microarray-based gene expression analysis. See for example, Shamir & Sharan (2002) Algorithmic approaches to clustering gene expression data. In Current Topics In Computational Molecular Biology (Edited by: Jiang T, Xu Y, Smith T). 2002, 269-300; Tamames et al., (2002): Bioinformatics methods for the analysis of expression arrays: data clustering and information extraction, J Biotechnol, 98:269-283.

### Dosage

The dose administered to an animal, particularly a human, in accordance with the present invention should be sufficient to effect the desired response in the animal over a reasonable time frame. One skilled in the art will recognize that dosage will depend upon a variety of factors, including the strength of the particular compositions employed, the age, species, condition, and body weight of the animal. The size of the dose also will be determined by the route, timing and frequency of administration as well as the existence, nature, and extent of any adverse side effects that might accompany the administration of a particular composition and the desired physiological effect. It will be appreciated by one of ordinary skill in the art that various conditions or desired results, may require prolonged treatment involving multiple administrations.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached.

The amount of the compound of the invention administered per dose or the total amount administered per day may be predetermined or it may be determined on an individual patient basis by taking into consideration numerous factors, including the nature and severity of the patient's condition, the condition being treated, the age, weight, and general health of the patient, the tolerance of the patient to the compound, the route of administration, pharmacological considerations such as the activity, efficacy, pharmacokinetics and toxicology profiles of the compound and any secondary agents being administered, and the like. Patients undergoing such treatment will typically be monitored on a routine basis to determine the effectiveness of therapy. Continuous monitoring by the physician will insure that the optimal amount of the compound of the invention will be administered at any given time, as well as facilitating the determination of the duration of treatment. This is of particular value when secondary agents are also being administered, as their selection, dosage, and duration of therapy may also require adjustment. In this way, the treatment regimen and dosing schedule can be adjusted over the course of therapy so that the lowest amount of compound that exhibits the desired effectiveness is administered and, further, that administration is continued only so long as is necessary to successfully achieve the optimum effect.

### DETAILED DESCRIPTION

Disclosed herein are new biomarkers associated with polyamine transport and cell lines expressing high levels of specific proteins provided herein are shown to have high polyamine transport activity and to be especially sensitive to therapies which target polyamine transport and polyamine metabolism. This property is shown in several assays where cell lines containing active polyamine transport were specifically targeted by compounds which utilize the polyamine transport system for cell entry. Therefore, the inventors have discovered herein, new specific biomarkers wherein their relative expression can be used to identify tumors (and patients) who will best respond to therapies which target cell growth processes which rely on polyamines, i.e., polyamine metabolism. The discovery of a new polyamine transport gene has provided opportunities to develop diagnostic tests to allow more directed and specific treatment of cancers and other proliferative disorders with drugs that target polyamine metabolism. Various assays and test kits can be used as described herein to rapidly identify and assess the relative expression of these biomarkers in biopsies of patient tissue, tumors, or other patient derived samples such as blood, urine, feces, tears, etc. These tests can be used in clinical labs, for example, or may be used in a healthcare provider office setting, or for at-home use in other, non-limiting embodiments.

Assays, therefore, which allow one to conveniently determine the relative expression of these biomarkers will assist clinicians performing clinical trials on polyamine based drugs or drugs that target polyamine metabolism. This technology forms the basis of a new personalized medicine where one can analyze a patient sample for these specific markers and know that that patient will respond to the polyamine targeted therapy. This will reduce the number of patients subjected to medications which will not help their specific cancer or disease state. Assays that may be used to determine presence of a particular marker in a biological sample include, but are not limited to, immunoassay, spectrometry, mass spectrometry, Matrix Assisted Laser Desorption/Ionization Time-of-Flight (MALDI-TOF) Mass Spectrometry, microscopy, northern blot, western blot, southern blot, isoelectric focusing, SDS-PAGE, PCR, RT-PCR, gel electrophoresis, protein microarray, DNA microarray, antibody microarray, ELISA, and combinations thereof.

Despite more than two decades of study, the bulk of the macromolecules involved in polyamine transport and the details of polyamine trafficking events within the cell remain elusive until the discovery of a particular biomarker described herein. In addition to improving our understanding of a fundamental biological process, knowledge of how polycationic materials are imported and sequestered in eukaryotic cells can lead to new drug-delivery and gene-delivery technologies that are selective against malignant cells and infectious protozoan parasites (Gardner, R. A.; Delcros, J-G.; Konate, F.; Breitbeil III, F.; Martin, B.; Sigman, M.; Huang, M.; Phanstiel IV, O. N¹-Substituent Effects in the Selective Delivery of Polyamine-Conjugates into Cells Containing Active Polyamine Transporters. J. Med. Chem. 2004, 47, 6055-6069; Gardner, R. A.; Belting, M.; Svensson, K.; Phanstiel IV, O. Synthesis and transfection efficiencies of new lipophilic polyamines. J. Med. Chem. 2007, 50, 308-318; Blagbrough I. S.; Geall A. J. Homologation of polyamines in the synthesis of lipo-spermine conjugates and related lipoplexes. Tetrahedron Lett. 1998, 39, 443-446; Azzam T.; Eliyahu H.; Shapira L.; Linial M.; Barenholz Y.; Domb A. J. Polysaccharide-oligoamine based conjugates for gene delivery. J. Med. Chem. 2002, 45, 1817-1824).

C-Myc has been shown to be overexpressed in pancreatic cancers (both primary and metastatic) and was thought in 2002 to be “involved in early neoplastic development and progression rather than in locoregional spread of invasive cancer.” (Ref: C. Schleger, Ph.D., C. Verbeke, M. D., R. Hildenbrand, M. D., H. Zentgraf, Ph.D., U. Bleyl, M. D. c-MYC Activation in Primary and Metastatic Ductal Adenocarcinoma of the Pancreas: Incidence, Mechanisms, and Clinical Significance. Modern Pathology 2002, 15, 462-469) However, a 2011 review paper on the role of c-Myc in pancreatic cancer (Ref: Anouchka Skoudy, Inmaculada Hernandez-Muñoz, Pilar Navarro. Pancreatic Ductal Adenocarcinoma and Transcription Factors: Role of c-Myc. J Gastrointest Canc 2011, 42, 76-84.) stated that c-Myc function is highly dose and cell dependent and that c-Myc has the ability to reprogram somatic cells towards a pluripotent stem like state. This means that therapies which can reduce c-Myc and early neoplastic growth could provide effective therapies against pancreatic and other cancers. In this regard c-myc is a known oncogene which increases the transcription of polyamine biosynthetic genes like ODC (Casero, Jr., R. and Marton, L. Targeting polyamine metabolism and function in cancer and other hyperproliferative diseases. Nature Reviews, 2007, 6, 373-390.)

The discovery of CATP-5 as an ATPase involved in polyamine transport in C. elegans (i.e., worms) provided clues as to the Drosophila and human genes via sequence similarities. (Heinick A, Urban K, Roth S, Spies D, Nunes, F Phanstiel O 4th, Liebau E, Luersen K. Caenorhabditis elegans P5B-type ATPase CATP-5 operates in polyamine transport and is important for norspermidine-mediated suppression of RNA interference. FASEB J. 2010, 24:206-217.) This area of polyamine transport was recently reviewed by Poulin R, Casero R A, Soulet D, where the possibility of ATPases playing a role in human polyamine transport were suggested. See Poulin et al., Recent advances in the molecular biology of metazoan polyamine transport. Amino Acids. 2012, 42, 711-723.

Flamigni has suggested the existence of an oncogene-polyamine metabolic loop, where the products of oncogenes like Kras and Raf result in downstream proteins c-Myc and AP-1, which regulate ODC transcription and therefore polyamine biosynthesis. Note: AP-1 is either a homodimer of Jun or a heterodimer of Jun and Fos, which bind to a common DNA binding site located in introns 3, 5, and 11 of the ODC gene. The Myc protein, on the other hand, is a transcription factor that regulates the expression of genes by binding to the specific DNA sequence CACGTG. Among the target genes for Myc regulation is ODC. Both Myc and AP-1 are substrates of phosphorylation by MAPK. (Flamigni F, Facchini A, Capanni C, Stefanelli C, Tantini B, and Caldarera C M. p44/42 Mitogen-activated protein kinase is involved in the expression of ornithine decarboxylase in leukemia L1210 cells. Biochem J. 1999, 341, 363-369. And see Uriel Bachrach, Yong-Chun Wang, and Amalia Tabib. Polyamines: New Cues in Cellular Signal Transduction. News Physiol. Sci. 2001, 16, 106-109.

Therefore, strategies which result in decreased intracellular polyamine content (reduced levels of Spermidine and putrescine are known to be caused by DFMO+PTI therapies, see Muth A., Madan M., Archer J. J., Ocampo N., Rodriguez L., Phanstiel O. 4th. Polyamine transport inhibitors: design, synthesis, and combination therapies with difluoromethylornithine. J Med Chem. 2014, 57, 348-363) as well as reduced c-Myc expression (as seen with DFMO and DFMO+GW5074 (FIG. 5, herein) will halt cell growth as the polyamine signaling molecules are depleted within cells due to an inability to make them and a blockade of their import. Since Myc has been linked to polyamine transport, therapies which modulate Myc expression may have an impact on polyamine transport and cell proliferation as well (Ref: Chang, B. K.; Libby, P. R.; Bergeron, R. J.; Porter, C. W. Modulation of polyamine biosynthesis and transport by oncogene transfection. Biochem Biophys Res Commun. 1988, 157, 264-270). By measuring particular biomarkers indicative of polyamine activity, one can assess the level of a tumor's commitment or reliance upon polyamine import processes as a means to supply its polyamine pools needed for its growth.

It has been discovered that certain key proteins are associated with increased polyamine import in mammalian cells. These proteins include: c-myc, Cav-1 and ATP13A3 (also referred to herein as PTS markers or biomarkers). Specifically, in pancreatic cancer cells, for example, the inventors have observed that cells with high expression of c-myc and/or ATP13A3 and low Cav-1 expression have high polyamine transport activity. Moreover, ATP13A3 and its role in polyamine transport was unknown until its discovery by the inventors as provided herein.

It has been identified herein that human pancreatic cancer cell lines which have high c-myc expression have high basal polyamine import rates as measured by the uptake of radiolabeled spermidine (high Vmax values). In this regard, high c-myc expression (as measured by Western blot and c-myc protein/beta-actin protein ratios) is a good indicator of a cell's high polyamine import rates. Thus, tumors with high c-myc expression would be expected to be sensitive to compounds which enter and kill cells via the polyamine transport system.

It has been observed that the expression of ATP13A3 increases when cells are treated with a polyamine biosynthesis inhibitor, difluoromethylornithine (DFMO) and that spermidine can modulate the levels of this ATP13A3 protein. These observations are consistent with ATP13A3 playing a role in polyamine transport. Indeed, the related protein, CATP-5, in the C. elegans model system was shown to play a role in polyamine transport. In addition, cells with high basal ATP13A3 expression have been shown to readily escape DFMO therapy via their increased import of exogenous spermidine. In this regard, ATP13A3 is an indicator as to the potential of the cell type or tumor to escape DFMO only therapy. This finding is important as ATP13A3 indicates the ability of cancer cells to escape DFMO via polyamine import. In short, cells or tumors with high ATP13A3 expression are predicted to be good at escaping DFMO-only therapy via their enhanced ability to import exogenous spermidine. These cells are still sensitive to a combination therapy (DFMO+PTI) involving DFMO and a polyamine transport inhibitor (PTI), where this escape pathway has been blocked via the PTI. In short, higher doses of the PTI have been shown to be able to overcome this increased ATP13A3 expression by blocking polyamine import in vitro. Thus, ATP13A3 represents a good biomarker for polyamine uptake and ultimately DFMO resistance.

The discoveries described herein are critical for the development of new cancer therapies predicated upon targeting polyamine transport and inducing sustained intracellular polyamine depletion. In a non-limiting embodiment, these cancer therapies may be accomplished using a combination therapy involving DFMO and a PTI (Burns, M. R.; Graminski, G. F.; Weeks, R. S.; Chen, Y.; O'Brien, T. G. Lipophilic Lysine-Spermine Conjugates Are Potent Polyamine Transport Inhibitors for Use in Combination with a Polyamine Biosynthesis Inhibitor, J Med Chem 2009, 52, 1983-1993; Aaron Muth, Jennifer Archer, Nicolette Ocampo, Meenu Madan, Luis Rodriguez, and Otto Phanstiel IV, Polyamine transport inhibitors: Design, Synthesis and Combination therapies with Difluoromethylornithine, J. Med. Chem. 2014, 57, 348-363).

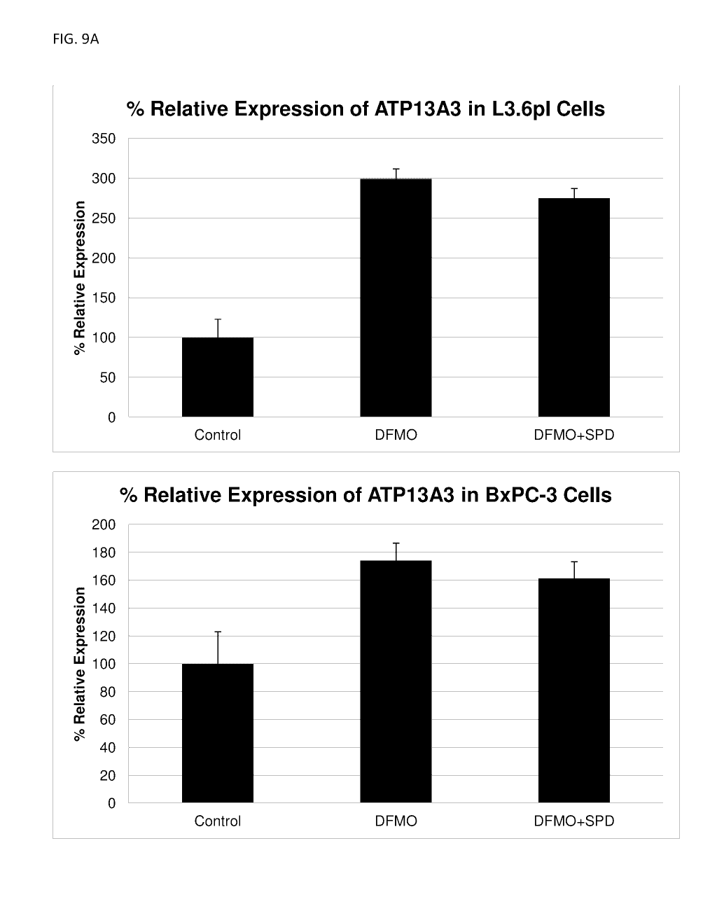

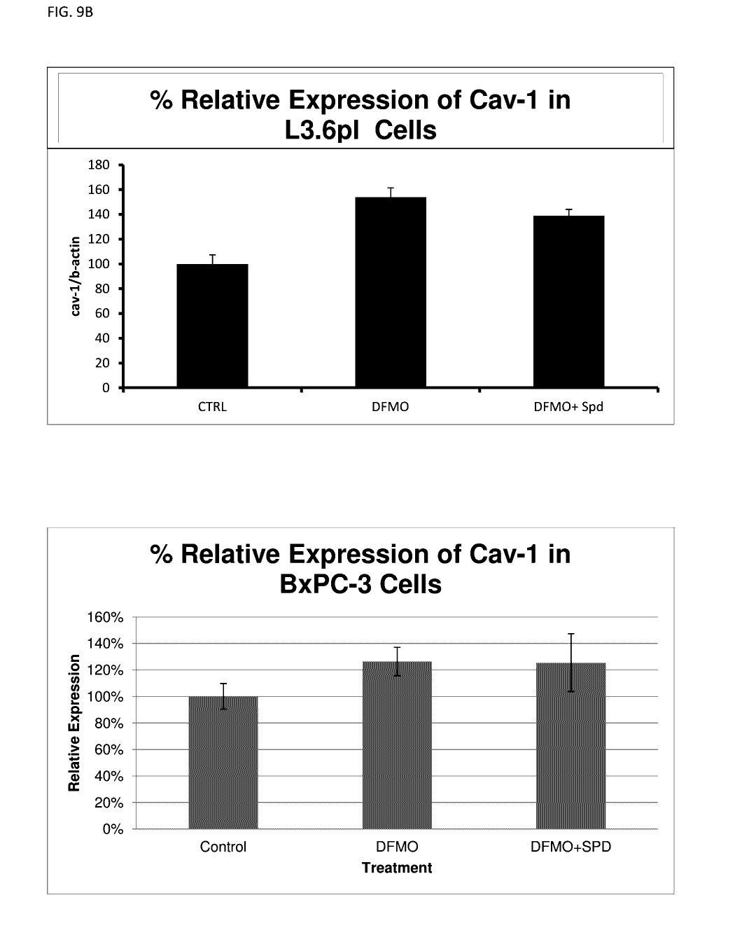

Caveolin-1 (Cav-1) levels also provide a predictive tool for how tumors may respond to DFMO therapy. For example, DFMO is expected to lower intracellular Cav-1 levels as well as Cav-1 mRNA (Altaf Mohammed, Naveena B. Janakiram, Venkateshwar Madka, Rebekah L. Ritchie, Misty Brewer, Laura Biddick, Jagan Mohan R. Patlolla, Michael Sadeghi, Stan Lightfoot, Vernon E. Steele, and Chinthalapally V. Rao* Eflornithine (DFMO) Prevents Progression of Pancreatic Cancer by Modulating Ornithine Decarboxylase Signaling, Cancer Research Prevention 2014, in press, doi: 10.1158/1940-6207. CAPR-14-0176). This outcome however may be cell line dependent as shown in FIG. 9B.

Recently, DFMO has also been shown to affect thymidine metabolism and affect thymidine levels in cancer cells. (Witherspoon, M., et al Cancer Discov; 2013, 3(9); 1072-1081). In this regard, DFMO therapy may affect other metabolic cycles linked to polyamine metabolism, some of which may adversely affect cell growth. One endpoint that can be considered in DFMO+PTI therapy is intracellular thymidine and SAM levels, which are expected to decrease as cells futilely attempt to generate decarboxylated SAM for polyamine biosynthesis. These endpoints could be used to provide a direct measure of therapy efficacy.

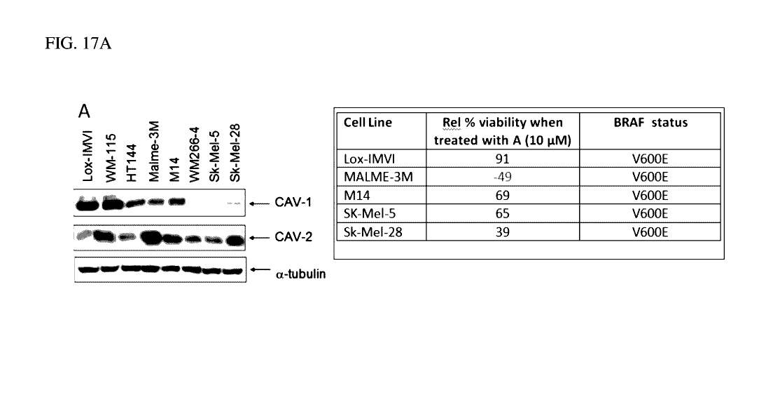

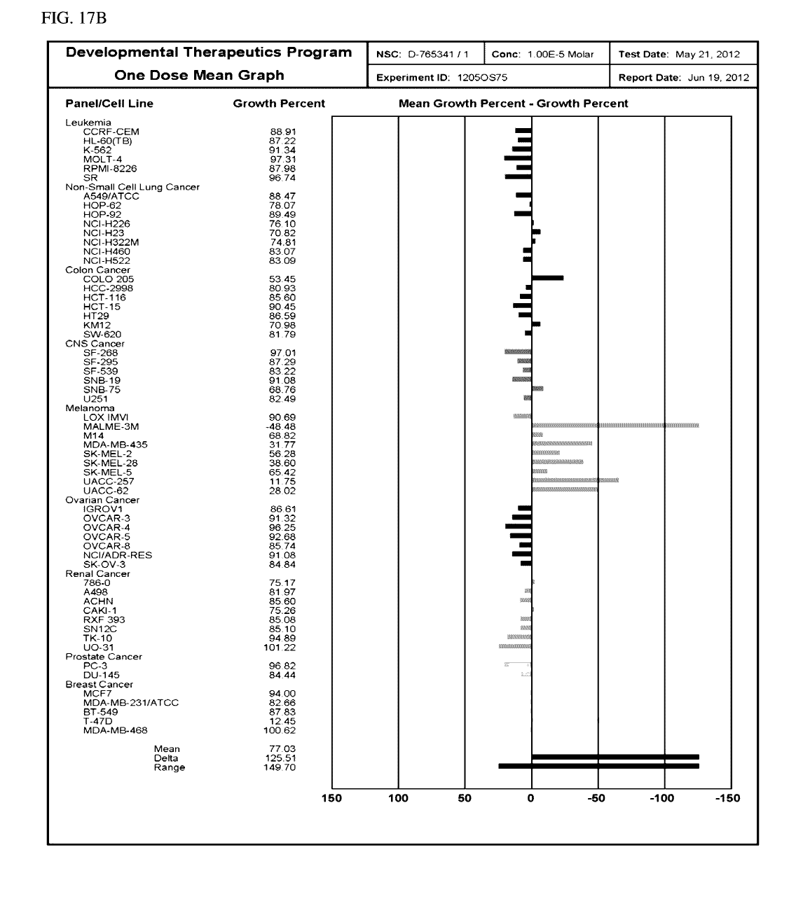

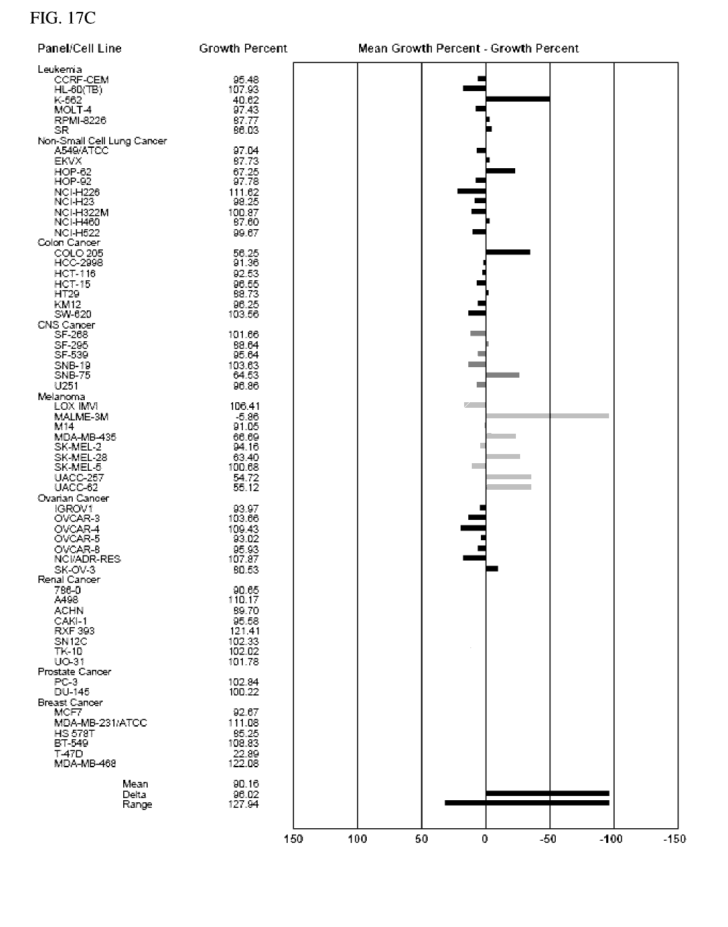

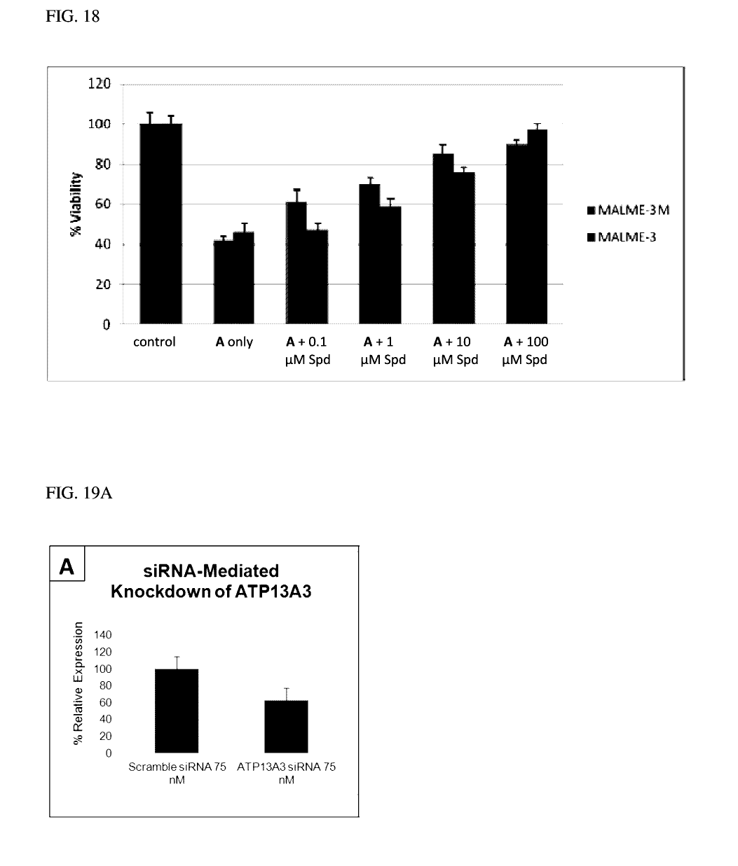

It is disclosed that measuring relative c-myc expression levels as well as Cav-1 and ATP13A3 levels provides a critical step in understanding to what degree a tumor is committed to polyamine import as a means to supply its polyamine pools needed for growth, and therefore, the potential for effectiveness of polyamine targeting drugs on the tumor. Measuring relative Cav-2 levels has also provided important information in determining to what degree the tumor is committed to polyamine import for growth, and the effectiveness of polyamine targeting drugs on these cells. As shown in FIG. 18A, the Cav2/Cav1 ratio correlated with the sensitivity of a series of melanoma cell lines to the polyamine transport targeting compound, compound A (FIG. 17).

Providing a convenient assay to specifically look at these markers will have tremendous clinical utility in helping physicians understand the tumors they are dealing with and to prescribe effective therapies which take advantage of this phenotype of high polyamine transport activity by ferrying in toxic polyamine based compounds (like compound A), or blocking the uptake of polyamine growth factors with DFMO+PTI, etc. In short, this discovery can help inform clinicians as to the nature of the tumors they seek to treat and provide additional confidence that the polyamine-based intervention will be successful.

Examples of biological samples typically obtained for use in certain method embodiments include but are not limited to a fluid, cell, stool sample, tissue, or tissue lysate obtained from the subject. Examples of fluids include but are not limited blood, serum, semen, urine, saliva, tears, perspiration, breath condensate, or vaginal fluid, cerebral spinal fluid, epithelial mucus or synovial fluid. Biological samples can be obtained from a subject, which may be a human or non-human mammal.

In non-limiting embodiments provided herein, a high or higher level of a biomarker includes a level at least 50% higher or at least two times the 50% higher level relative to basal levels in normal cells. In non-limiting embodiments provided herein basal level refers to the level in normal cells. In other non-limiting embodiments provided herein, a lower level of a biomarker includes a level within 20% of the basal level.

### Assay and Diagnostic Testing Methods

Embodiments of the invention include diagnostic tests, testing methods and assays to determine the presence of certain PTS markers disclosed herein. Various types of assays and tests may be used, including, but not limited to immunoassays, spectrometry, mass spectrometry, Matrix Assisted Laser Desorption/Ionization Time-of-Flight (MALDI-TOF) Mass Spectrometry, microscopy, northern blot, western blot, southern blot, isoelectric focusing, SDS-PAGE, PCR, RT-PCR, gel electrophoresis, protein microarray, DNA microarray, and antibody microarray, or combinations thereof. Preferably, the level of the one or more biomarkers is determined using an immunoassay. An immunoassay uses an antibody or antibodies to a specific antigen to determine the levels of the antigen.

The immunoassay may be an enzyme linked immunoassay (ELISA), a sandwich assay, a radioimmunoassay, a Western Blot, an immunoassay using a biosensor, an immunoprecipitation assay, an agglutination assay, a turbidity assay or a nephelometric assay.

The one or more antibodies may be synthetic, monoclonal, polyclonal, bispecific, chimeric or humanised. A chimeric antibody includes portions derived from different animals. Humanised antibodies are antibodies from non-human species having one or more complementarity determining regions from the non-human species and a framework region from a human immunoglobulin molecule. Chimeric and humanised antibodies can be produced by recombinant techniques well known in the art.

The one or more antibodies may comprise a tag or a label selected from the group comprising a radioactive, a fluorescent, a chemiluminescent, a dye, an enzyme, or a histidine tag or label, or any other suitable label or tag known in the art.

The use of subscript A, B, (e.g. Antibody_{A}, Antibody_{B}, etc.) unless otherwise indicated, is an arbitrary designation to indicate that they are different antibodies, or antibodies that are each specific to a different protein.

The presence, and possibly even the level, of particular proteins in a sample may also be determined by using mass spectrometry techniques. Mass spectrometry techniques may be used to detect gas phase ions which correlate to specific proteins or parts of proteins, such as, trypsin peptides. Examples of mass spectrometers include time of flight, magnetic sector, quadruple filter, ion trap, ion cyclotron resonance, electrostatic sector analyser and hybrids of these. The mass spectrometer may use laser desorption/ionisation.

MALDI (Matrix-assisted laser desorption/ionization) peptide mass fingerprinting may be used to identify the presence of particular biomarker proteins from 2D gel analysis and RPE/SDS PAGE analysis.

MALDI-MS and ANN analysis may be used to profile identified proteins and tryptic biomarker signatures to determine that certain cancer cells are susceptible to PTS targeting.

In a particular embodiment, disclosed is the use of immobilized antibodies which target and identify c-myc, ATP13A3, Cav-1 or Cav-2 proteins, or a combination thereof. These could be in the form of an ELISA test, in a non-limiting embodiment, which may quantify the relative expression levels of ATP13A3, c-myc Cav-1 and/or Cav-2. High expression of ATP13A3, Cav-2 and c-myc would indicate high polyamine transport activity by the tumor (or sample), in one embodiment. Low Cav-1 expression would indicate increased polyamine transport activity, in an embodiment. In a further embodiment, low Cav-1 expression where c-myc, Cav-2, and/or ATP13A3 activity is high could indicate increased polyamine transport activity. In a further embodiment, low Cav-1 expression in samples where ATP13A3 expression is high could indicate increased polyamine transport activity in the presence of DFMO and indicate a higher level of PTI dosing to ensure efficacy. Further, low Cav-1 expression in samples where Cav-2 expression is high could indicate sensitivity to polyamine transport inhibitors like trimer44NMe or other polyamine transport targeting compounds, such as, in non-limiting examples, Compounds A and/or B as described in more detail herein. Endpoints could be measured in terms of tumor size, weight and histological examination of tumor biopsies looking for these markers by immunohistochemistry (IHC) or by Western blot analysis using a standard protein like beta actin or GADPH for normalization and quantification.

The ability to easily quantify these markers would provide guidance for treatment using effective and specific treatment therapies, including many polyamine targeting therapies like DFMO alone or in combinations with NSAIDs, for example, or smart drugs which selectively target cells with active polyamine transport (Ant44 and compounds A and B) (Development of Polyamine Transport Ligands with Improved Metabolic Stability and Selectivity against Specific Human Cancers. Aaron Muth, Joseph Kamel, Navneet Kaur, Allyson C. Shicora, Iraimoudi S. Ayene, Susan K. Gilmour, and Otto Phanstiel IV J. Med. Chem. 2013, 56, 5819-5828.), in non-limiting embodiments. In other non-limiting embodiments, combination therapies which use a polyamine biosynthesis inhibitor (e.g., DFMO) in concert with a polyamine transport inhibitor (PTI) may be used to target these cells. (Polyamine transport inhibitors: Design, Synthesis and Combination therapies with Difluoromethylornithine, Aaron Muth, Jennifer Archer, Nicolette Ocampo, Meenu Madan, Luis Rodriguez, and Otto Phanstiel IV. J. Med. Chem. 2014, 57, 348-363.) All of these medical interventions will be more effective against tumors with high expression of the biomarkers ATP13A3, c-myc, and/or Cav-2 (and in some instances concomitant lower expression of Cav-1). A multitude of therapies can be enhanced by the pre-selection of patients (and/or tumors) who will best respond to the therapies identified with the tests and methods described herein.

In Table A below, therapeutic suggestions based on biomarker levels are provided in non-limiting examples. For example, high ATP13A3 and low Cav-1 levels are indicative of high polyamine transport, and consequently, treatment with DFMO-only would be less effective than a combination treatment with DFMO and PTI. Low Cav-1 and high ATP13A3 indicate high potential of that cell to escape DFMO therapy via polyamine import. Consequently effective treatment strategies would include increased PTI to overcome the increased transport activity, for example (see Table A).

TABLE A

| | basal level | expected level | | | Implications |
|---|---|---|---|---|---|
| | relative | in the presence | Outcome with | | for DFMO + |
| Marker | expression | of DFMO therapy | DFMO therapy | basal level Indications | PTI therapy |
| Cav-1 | high | | | high Cav-1 indicates | |
| | | | | low potential of that | |
| | | | | cell to escape DFMO | |
| | | | | therapy via polyamine | |
| | | | | import | |
| Cav-1 | low | Same or | Either the same or | low Cav-1 indicates | need to add more |
| | | significantly | increased polyamine | high potential of that | PTI to overcome the |
| | | lower | import if Cav-1 | cell to escape DFMO | increased transport |
| | | | levels are decreased | therapy via polyamine | activity |
| | | | | import | |
| ATP13A3 | high | significantly | increased | high ATP13A3 | need to add more |
| | | increased | polyamine | indicates high potential | PTI to overcome the |
| | | | import | of that cell to escape | increased transport |
| | | | | DFMO therapy via | activity |
| | | | | polyamine import | |
| ATP13A3 | low | | | low ATP13A3 | |
| | | | | indicates low potential | |
| | | | | of that cell to escape | |
| | | | | DFMO therapy via | |
| | | | | polyamine import | |
| c-Myc | high | decreased | | high c-Myc indicates | need to add more |
| | | | | high polyamine | PTI to overcome the |
| | | | | transport activity, | increased transport |
| | | | | should be sensitive to | activity |
| | | | | polyamine transport | |
| | | | | targeting compounds, | |
| | | | | e.g., Ant44 | |
| c-Myc | low | | | low c-Myc indicates | |
| | | | | low polyamine | |
| | | | | transport activity | |
| c-Raf | high | | | high polyamine | need to add more |
| | | | | transport activity | PTI to overcome the |
| | | | | | increased transport |
| | | | | | activity |
| c-Raf | low | | | reduced polyamine | |
| | | | | transport activity | |

While protein levels can be used for this detection purpose, other biomolecules like RNA and DNA which encode for these specific proteins can also be used. This broadens the application to include detection methods for these nucleic acid sequences which correspond to the listed protein biomarkers (e.g., ATP13A3, Cav-2, etc).

Quantitative polymerase chain reaction (QPCR) tests may be used, in another embodiment, to measure the relative gene expression of the PTS markers described herein. Both DNA (QPCR) and RNA (via reverse transcriptase and RT-PCR) can be measured by this method to provide relative mRNA and DNA levels of each of these genes in cells/tumors. This method is very sensitive and has real cost saving value as there exist commercial instruments to perform these routine experiments.

DNA microarray may also be used to quantify the relative gene expression of these PTS markers. A DNA microarray (also commonly known as DNA chip or biochip) is a collection of microscopic DNA spots attached to a solid surface. Scientists use DNA microarrays to measure the expression levels of large numbers of genes simultaneously or to genotype multiple regions of a genome. Each DNA spot may contain picomoles (10⁻¹² moles) of a specific DNA sequence, known as probes (or reporters or oligos). These can be a short section of a gene or other DNA element that are used to hybridize a cDNA or cRNA (also called anti-sense RNA) sample (called target) under high-stringency conditions. Probe-target hybridization is usually detected and quantified by detection of fluorophore-, silver-, or chemiluminescence-labeled targets to determine relative abundance of nucleic acid sequences in the target.

Other examples for identifying these PTS markers may include any process of assessing protein expression including flow cytometry, immunohistochemistry, immunocytochemistry, ELISA, Western blot, and immunoaffinity chromatography, HPLC, mass spectrometry, protein microarray analysis, PAGE analysis, isoelectric focusing, 2-D gel electrophoresis, or any enzymatic assay.

Other methods used to assess expression include the use of natural or artificial ligands capable of specifically binding a marker. Such ligands include antibodies, antibody complexes, conjugates, natural ligands, small molecules, nanoparticles, or any other molecular entity capable of specific binding to a marker. Antibodies may be monoclonal, polyclonal, or any antibody fragment including an Fab, F(ab)₂, Fv, scFv, phage display antibody, peptibody, multispecific ligand, or any other reagent with specific binding to a marker. Ligands may be associated with a label such as a radioactive isotope or chelate thereof, dye (fluorescent or nonfluorescent,) stain, enzyme, metal, or any other substance capable of aiding a machine or a human eye from differentiating a cell expressing a marker from a cell not expressing a marker. Additionally, expression may be assessed by monomeric or multimeric ligands associated with substances capable of killing the cell. Such substances include protein or small molecule toxins, cytokines, pro-apoptotic substances, pore forming substances, radioactive isotopes, or any other substance capable of killing a cell.

Differential expression encompasses any detectable difference between the expression of a marker in one sample relative to the expression of the marker in another sample. Differential expression may be assessed by a detector, an instrument containing a detector, or by aided or unaided human eye. Examples include but are not limited to differential staining of cells in an IHC assay configured to detect a marker, differential detection of bound RNA on a microarray to which a sequence capable of binding to the marker is bound, differential results in measuring RTPCR measured in ΔCt or alternatively in the number of PCR cycles necessary to reach a particular optical density at a wavelength at which a double stranded DNA binding dye (e.g. SYBR Green) incorporates, differential results in measuring label from a reporter probe used in a real-time RTPCR reaction, differential detection of fluorescence on cells using a flow cytometer, differential intensities of bands in a Northern blot, differential intensities of bands in an RNAse protection assay, differential cell death measured by apoptotic markers, differential cell death measured by shrinkage of a tumor, or any method that allows a detection of a difference in signal between one sample or set of samples and another sample or set of samples.

The expression of the marker in a sample may be compared to a level of expression predetermined to predict the presence or absence of a particular physiological characteristic, or compared to levels of the biomarkers in normal cells. The level of expression may be derived from a single control or a set of controls. A control may be any sample with a previously determined level of expression. A control may comprise material within the sample or material from sources other than the sample. Alternatively, the expression of a marker in a sample may be compared to a control that has a level of expression predetermined to signal or not signal a cellular or physiological characteristic. This level of expression may be derived from a single source of material including the sample itself or from a set of sources. Comparison of the expression of the marker in the sample to a particular level of expression results in a prediction that the sample exhibits or does not exhibit the cellular or physiological characteristic.

Determining the level of expression that signifies a physiological or cellular characteristic may be assessed by any of a number of methods. The skilled artisan will understand that numerous methods may be used to select a level of expression for a particular marker or a plurality of markers that signifies a particular physiological or cellular characteristic. In diagnosing the presence of a disease, a threshold value may be obtained by performing the assay method on samples obtained from a population of patients having a certain type of disease (cancer for example) and from a second population of subjects that do not have the disease. In assessing disease outcome or the effect of treatment, a population of patients, all of which have, a disease such as cancer, may be followed for a period of time. After the period of time expires, the population may be divided into two or more groups. For example, the population may be divided into a first group of patients whose disease progresses to a particular endpoint and a second group of patients whose disease does not progress to the particular endpoint. Examples of endpoints include disease recurrence, death, metastasis or other states to which disease may progress. If expression of the marker in a sample is more similar to the predetermined expression of the marker in one group relative to the other group, the sample may be assigned a risk of having the same outcome as the patient group to which it is more similar.

Cancer cells include any cells derived from a tumor, neoplasm, cancer, pre-cancer, cell line, malignancy, or any other source of cells that have the potential to expand and grow to an unlimited degree. Cancer cells may be derived from naturally occurring sources or may be artificially created. Cancer cells may also be capable of invasion into other tissues and metastasis when placed into an animal host. Cancer cells further encompass any malignant cells that have invaded other tissues and/or metastasized. One or more cancer cells in the context of an organism may also be called a cancer, tumor, neoplasm, growth, malignancy, or any other term used in the art to describe cells in a cancerous state.

According to other embodiments, provided are kits designed for determining PTS markers in a biological sample. The kit may further include one or more of a buffer solution, a conjugating solution for developing the signal of interest, or a detection reagent for detecting the signal of interest, each in separate packaging, such as a container. In another example, the kit includes a plurality of size-associated marker of target nucleic acid sequences for hybridization with a detection array. The kit can also include instructions in a tangible form, such as written instructions or in a computer-readable format.

Kits comprising a primer or probe that is complementary to and specifically hybridizes to or binds to a target genes/mRNA in a sample and enzymes suitable for amplifying target genes/mRNA are provided in certain embodiments of the invention. The primer or probe may be labeled with a radioisotope, a fluorophore, a chromophore, a dye, an enzyme, or TOF carrier. In these kits, binding may be detected by in situ hybridization, PCR RT-PCR, fluorescence resonance energy transfer, chemiluminescence enzymatic signal amplification, electron dense particles magnetic particles and capacitance coupling. The probe is selected to allow the target genes/mRNA to be sequenced if wanted, or for quantitation of the respective different target genes/mRNA as compared to the wild-type sequence. These reagents in certain embodiments may comprise one or more nucleic acid probes, may be in the form of a microarray, are suitable for primer extension and can comprise controls indicative of a healthy individual.

Nucleotides may be amplified to obtain amplification products. Suitable nucleic acid amplification techniques are well known to a person of ordinary skill in the art, and include polymerase chain reaction (PCR) as for example described in Ausubel et al., Current Protocols in Molecular Biology (John Wiley & Sons, Inc. 1994-1998) (and incorporated herein by reference). The most commonly used nucleic acid amplification technique is the polymerase chain reaction (PCR).

PCR is well known in this field and comprehensive description of this type of reaction is provided in E. van Pelt-Verkuil et al., Principles and Technical Aspects of PCR Amplification, Springer, 2008. PCR is a powerful technique that amplifies a target DNA sequence against a background of complex DNA. If RNA is to be amplified (by PCR), it must be first transcribed into cDNA (complementary DNA) using an enzyme called reverse transcriptase. Thereafter, the resulting cDNA is amplified by PCR. PCR is an exponential process that proceeds as long as the conditions for sustaining the reaction are acceptable. The components of the reaction are: i. pair of primers—short single strands of DNA with around 10-30 nucleotides complementary to the regions flanking the target sequence (typically the reporter nucleotide of the marker complex); ii. DNA polymerase—a thermostable enzyme that synthesizes DNA; iii. deoxyribonucleoside triphosphates (dNTPs)—to provide the nucleotides that are incorporated into the newly synthesized DNA strand; and iv. buffer—to provide the optimal chemical environment for DNA synthesis.

PCR typically involves placing these reactants in a small tube (˜10-50 μL) containing the extracted nucleic acids. The tube is placed in a thermal cycler; an instrument that subjects the reaction to a series of different temperatures for varying amounts of time. The standard protocol for each thermal cycle involves a denaturation phase, an annealing phase, and an extension phase. The extension phase is sometimes referred to as the primer extension phase. In addition to such three-step protocols, two-step thermal protocols can be employed, in which the annealing and extension phases are combined. The denaturation phase typically involves raising the temperature of the reaction to 90-95° C. to denature the DNA strands; in the annealing phase, the temperature is lowered to 50-60° C. for the primers to anneal; and then in the extension phase the temperature is raised to the optimal DNA polymerase activity temperature of 60-72° C. for primer extension. This process is typically repeated cyclically around 20-40 times, in one example.

There are a number of variants to the standard PCR protocol such as multiplex PCR, linker-primed PCR, direct PCR, tandem PCR, real-time PCR, and reverse-transcriptase PCR, amongst others, which have been developed for molecular diagnostics.

Multiplex PCR uses multiple primer sets within a single PCR mixture to produce amplicons of varying sizes that are specific to different DNA sequences. By targeting multiple genes at once, additional information may be gained from a single test-run that otherwise would require several experiments. Optimization of multiplex PCR is more difficult though and requires selecting primers with similar annealing temperatures, and amplicons with similar lengths and base composition to ensure the amplification efficiency of each amplicon is equivalent.

Linker-primed PCR, also known as ligation adaptor PCR, is a method used to enable nucleic acid amplification of essentially all DNA sequences in a complex DNA mixture without the need for target-specific primers. The method firstly involves digesting the target DNA population with a suitable restriction endonuclease (enzyme). Double-stranded oligonucleotide linkers (also called adaptors) with a suitable overhanging end are then ligated to the ends of target DNA fragments using a ligase enzyme. Nucleic acid amplification is subsequently performed using oligonucleotide primers which are specific for the linker sequences. In this way, all fragments of the DNA source, which are flanked by linker oligonucleotides, can be amplified.

Direct PCR describes a system whereby PCR is performed directly on a sample without any, or with minimal, nucleic acid extraction. It has long been accepted that PCR reactions are inhibited by the presence of many components of unpurified biological samples, such as the heme component in blood. Traditionally, PCR has required extensive purification of the target nucleic acid prior to preparation of the reaction mixture. With appropriate changes to the chemistry and sample concentration, however, it is possible to perform PCR with minimal DNA purification, or direct PCR. Adjustments to the PCR chemistry for direct PCR include increased buffer strength, the use of polymerases which have high activity and processivity, and additives.

Tandem PCR utilizes two distinct rounds of nucleic acid amplification to increase the probability that the correct amplicon is amplified. One form of tandem PCR is nested PCR in which two pairs of PCR primers are used to amplify a single locus in separate rounds of nucleic acid amplification. The first pair of primers hybridize to the nucleic acid sequence at regions external to the target nucleic acid sequence. The second pair of primers (nested primers) used in the second round of amplification bind within the first PCR product and produce a second PCR product containing the target nucleic acid, that will be shorter than the first one. The logic behind this strategy is that if the wrong locus were amplified by mistake during the first round of nucleic acid amplification, the probability is very low that it would also be amplified a second time by a second pair of primers and thus ensures specificity.

Real-time PCR, or quantitative PCR, is used to measure the quantity of a PCR product in real time. By using a fluorophore-containing probe or fluorescent dyes along with a set of standards in the reaction, it is possible to quantitate the starting amount of nucleic acid in the sample. This is particularly useful in molecular diagnostics where treatment options may differ depending on the pathogen load in the sample.

If the reporter nucleotide is an RNA sequence, it can be amplified or converted into complementary DNA (cDNA), such as by using RT-PCR. Reverse-transcriptase PCR (RT-PCR) is used to amplify DNA from RNA. Reverse transcriptase is an enzyme that reverse transcribes RNA into cDNA, which is then amplified by PCR. RT-PCR is widely used in expression profiling, to determine the expression of a gene or to identify the sequence of an RNA transcript, including transcription start and termination sites. It is also used to amplify RNA viruses such as human immunodeficiency virus or hepatitis C virus. cDNA is DNA synthesized from a messenger RNA (mRNA) template in a reaction catalyzed by the enzyme reverse transcriptase and the enzyme DNA polymerase.

Frequently, the surface of a biochip comprises a plurality of addressable locations, each of which comprises a capture binding ligand. An “array location,” “addressable location,” “pad” or “site” herein means a location on the substrate that comprises a covalently attached capture binding ligand. An “array” herein means a plurality of capture binding ligands in a regular, ordered format, such as a matrix. The size of the array will depend on the composition and end use of the array. Arrays containing from about two or more different capture binding ligands to many thousands can be made. Generally, the array will comprise 3, 4, 5, 6, 7 or more types of capture binding ligands depending on the end use of the array. In the present invention, the array can include controls, replicates of the markers and the like. Exemplary ranges are from about 3 to about 50. In some embodiments, the compositions of the invention may not be in array format; that is, for some embodiments, compositions comprising a single capture ligand may be made as well. In addition, in some arrays, multiple substrates may be used, either of different or identical compositions. Thus for example, large arrays may comprise a plurality of smaller substrates.

Accordingly, in one aspect, the invention provides a composition comprising a solid support comprising a capture binding ligand for each biomarker of a biomarker panel. In various embodiments, the capture ligand is a nucleic acid. In various embodiments, the capture binding ligand is an antibody. In various embodiments, the composition further comprises a soluble binding ligand for each biomarker of a biomarker panel.

A number of different biochip array platforms as known in the art may be used. For example, the compositions and methods of the present invention can be implemented with array platforms such as GeneChip® (Affymetrix), CodeLink™ Bioarray (Amersham), Expression Array System (Applied Biosystems), SurePrint microarrays (Agilent), Sentrix® LD BeadChip or Sentrix® Array Matrix (Illumina) and Verigene (Nanosphere).

In various exemplary embodiments, detection and measurement of biomarkers utilizes colorimetric methods and systems in order to provide an indication of binding of a target analyte or target species. In colorimetric methods, the presence of a bound target species such as abiomarker will result in a change in the absorbance or transmission of light by a sample or substrate at one or more wavelengths. Detection of the absorbance or transmission of light at such wavelengths thus provides an indication of the presence of the target species.

A detection system for colorimetric methods includes any device that can be used to measure colorimetric properties as discussed above. Generally, the device is a spectrophotometer, a colorimeter or any device that measures absorbance or transmission of light at one or more wavelengths. In various embodiments, the detection system comprises a light source; a wavelength filter or monochromator; a sample container such as a cuvette or a reaction vial; a detector, such as a photoresistor, that registers transmitted light; and a display or imaging element.

In various exemplary embodiments, a ClonDiag chip platform is used for the colorimetric detection of biomarkers. In various embodiments, a ClonDiag ArrayTube (AT) is used. One unique feature of the ArrayTube is the combination of a micro probe array (the biochip) and micro reaction vial. In various embodiments, where a target sequence is a nucleic acid, detection of the target sequence is done by amplifying and biotinylating the target sequence contained in a sample and optionally digesting the amplification products. The amplification product is then allowed to hybridize with probes contained on the ClonDiag chip. A solution of a streptavidin-enzyme conjugate, such as Poly horseradish peroxidase (HRP) conjugate solution, is contacted with the ClonDiag chip. After washing, a dye solution such as o-dianisidine substrate solution is contacted with the chip. Oxidation of the dye results in precipitation that can be detected colorimetrically. Further description of the ClonDiag platform is found in Monecke S, Slickers P, Hotzel H et al., Clin Microbiol Infect 2006, 12: 718-728; Monecke S, Berger-Bächi B, Coombs C et al., Clin Microbiol Infect 2007, 13: 236-249; Monecke S, Leube I and Ehricht R, Genome Lett 2003, 2: 106-118; Monecke S and Ehricht R, Clin Microbiol Infect 2005, 11: 825-833; German Patent DE 10201463; US Publication US/2005/0064469 and ClonDiag, ArrayTube (AT) Experiment Guideline for DNA-Based Applications, version 1.2, 2007, all incorporated by reference in their entirety. One of skill in the art will appreciate that numerous other dyes that react with a peroxidase can be utilized to produce a colorimetric change, such as 3,3′,5,5′-tetramethylbenzidine (TMB). For information on specific assay protocols, see www.clondiag.com/technologies/publications.php.

In various embodiments, where a target species is a protein, the ArrayTube biochip comprises capture binding ligands such as antibodies. A sample is contacted with the biochip, and any target species present in the sample is allowed to bind to the capture binding ligand antibodies. A soluble capture binding ligand or a detection compound such as a horseradish peroxidase conjugated antibody is allowed to bind to the target species. A dye, such as TMB, is then added and allowed to react with the horseradish peroxidase, causing precipitation and a color change that is detected by a suitable detection device. Further description of protein detection using ArrayTube is found in, for example, Huelseweh B, Ehricht R and Marschall H-J, Proteomics, 2006, 6, 2972-2981; and ClonDiag, ArrayTube (AT) Experiment Guideline for Protein-Based Applications, version 1.2, 2007, all incorporated by reference in their entirety.

Transmission detection and analysis is performed with a ClonDiag AT reader instrument. Suitable reader instruments and detection devices include the ArrayTube Workstation ATS and the ATR 03.

In addition to ArrayTube, the ClonDiag ArrayStrip (AS) can be used. The ArrayStrip provides a 96-well format for high volume testing. Each ArrayStrip consists of a standard 8-well strip with a microarray integrated into the bottom of each well. Up to 12 ArrayStrips can be inserted into one microplate frame enabling the parallel multiparameter testing of up to 96 samples. The ArrayStrip can be processed using the ArrayStrip Processor ASP, which performs all liquid handling, incubation, and detection steps required in array based analysis. In various embodiments, where a protein is detected, a method of using the ArrayStrip to detect the protein comprises conditioning the AS array with buffer or blocking solution; loading of up to 96 sample solutions in the AS wells to allow for binding of the protein; 3× washing; conjugating with a secondary antibody linked to HRP; 3× washing; precipitation staining with TMB; and AS array imaging and optional data storage.

Those skilled in the art will be familiar with numerous additional immunoassay formats and variations thereof which may be useful for carrying out the method disclosed herein. See generally E. Maggio, Enzyme-Immunoassay, (CRC Press, Inc., Boca Raton, Fla., 1980); see also U.S. Pat. Nos. 4,727,022; 4,659,678; 4,376,110; 4,275,149; 4,233,402; and 4,230,767.

Antibodies

Antibodies for ATP13A3, Cav-1 and c-myc to use in the various detection assays described herein are commercially available. c-Myc Antibody (e10: (sc-40 Santa Cruz Biotechnology), ATP13A3 antibody (E-18) (sc102335, Santa Cruz Biotechnology). Caveolin-1 Antibody (7C8) (sc-53564, Santa Cruz Biotechnology). HPA-029471 is a Sigma polyclonal antibody that binds only to the intact ATP13A3 protein target. The ATP13A3 polyclonal antibody, HPA-029471, provides a band at approximately 120 kDa. In embodiments as described herein, HPA-029471 is used as the antibody to bind ATP13A3.

Sequences

Other known polyamine transport genes may also respond in this way and could be of value for tracking sample response to therapy including Oct2 and DAX.

Sequences of ATP13A3, Cav-1 c-myc and Cav-2 are provided below:

(SEQ ID No. 1)
MPLNVSFTNR NYDLDYDSVQ PYFYCDEEEN FYQQQQQSEL
QPPAPSEDIW KKFELLPTPP LSPSRRSGLC SPSYVAVTPF
SLRGDNDGGG GSFSTADQLE MVTELLGGDM VNQSFICDPD
DETFIKNIII QDCMWSGFSA AAKLVSEKLA SYQAARKDSG
SPNPARGHSV CSTSSLYLQD LSAAASECID PSVVFPYPLN
DSSSPKSCAS QDSSAFSPSS DSLLSSTESS PQGSPEPLVL
HEETPPTTSS DSEEEQEDEE EIDVVSVEKR QAPGKRSESG
SPSAGGHSKP PHSPLVLKRC HVSTHQHNYA APPSTRKDYP
AAKRVKLDSV RVLRQISNNR KCTSPRSSDT EENVKRRTHN
VLERQRRNEL KRSFFALRDQ IPELENNEKA PKVVILKKAT
AYILSVQAEE QKLISEEDLL RKRREQLKHK LEQLRNSCA
C-myc protein sequence
(SEQ ID NO. 2)
MSGGKYVDSE GHLYTVPIRE QGNIYKPNNK AMADELSEKQ
VYDAHTKEID LVNRDPKHLN DDVVKIDFED VIAEPEGTHS
FDGIWKASFT TFTVTKYWFY RLLSALFGIP MALIWGIYFA
ILSFLHIWAV VPCIKSFLIE IQCISRVYSI YVHTVCDPLF
EAVGKIFSNV RINLQKEI
Cav-1 polypeptide sequence
(SEQ ID No. 3)
MDREERKTIN QGQEDEMEIY GYNLSRWKLA IVSLGVICSG
GFLLLLLYWM PEWRVKATCV RAAIKDCEVV LLRTTDEFKM
WFCAKIRVLS LETYPVSSPK SMSNKLSNGH AVCLIENPTE
ENRHRISKYS QTESQQIRYF THHSVKYFWN DTIHNFDFLK
GLDEGVSCTS IYEKHSAGLT KGMHAYRKLL YGVNEIAVKV
PSVFKLLIKE VLNPFYIFQL FSVILWSTDE YYYYALAIVV
MSIVSIVSSL YSIRKQYVML HDMVATHSTV RVSVCRVNEE
IEEIFSTDLV PGDVMVIPLN GTIMPCDAVL INGTCIVNES
MLTGESVPVT KTNLPNPSVD VKGIGDELYN PETHKRHTLF
CGTTVIQTRF YTGELVKAIV VRTGFSTSKG QLVRSILYPK
PTDFKLYRDA YLFLLCLVAV AGIGFIYTII NSILNEVQVG
VIIIESLDII TITVPPALPA AMTAGIVYAQ RRLKKIGIFC
ISPQRINICG QLNLVCFDKT GTLTEDGLDL WGIQRVENAR
FLSPEENVCN EMLVKSQFVA CMATCHSLTK IEGVLSGDPL
DLKMFEAIGW ILEEATEEET ALHNRIMPTV VRPPKQLLPE
STPAGNQEME LFELPATYEI GIVRQFPFSS ALQRMSVVAR
VLGDRKMDAY MKGAPEAIAG LCKPETVPVD FQNVLEDFTK
QGFRVIALAH RKLESKLTWH KVQNISRDAI ENNMDFMGLI
IMQNKLKQET PAVLEDLHKA NIRTVMVTGD SMLTAVSVAR
DCGMILPQDK VIIAEALPPK DGKVAKINWH YADSLTQCSH
PSAIDPEAIP VKLVHDSLED LQMTRYHFAM NGKSFSVILE
HFQDLVPKLM LHGTVFARMA PDQKTQLIEA LQNVDYFVGM
CGDGANDCGA LKRAHGGISL SELEASVASP FTSKTPSISC
VPNLIREGRA ALITSFCVFK FMALYSIIQY FSVTLLYSIL
SNLGDFQFLF IDLAIILVVV FTMSLNPAWK ELVAQRPPSG
LISGALLFSV LSQIIICIGF QSLGFFWVKQ QPWYEVWHPK
SDACNTTGSG FWNSSHVDNE TELDEHNIQN YENTTVFFIS
SFQYLIVAIA FSKGKPFRQP CYKNYFFVFS VIFLYIFILF
IMLYPVASVD QVLQIVCVPY QWRVTMLIIV LVNAFVSITV
EESVDRWGKC CLPWALGCRK KTPKAKYMYL AQELLVDPEW
PPKPQTTTEA KALVKENGSC QIITIT
ATP13A3 Polypeptide sequence
(SEQ ID No. 4)
MGLETEKADV QLFMDDDSYS HHSGLEYADP EKFADSDQDR
DPHRLNSHLK LGFEDVIAEP VTTHSFDKVW ICSHALFEIS
KYVMYKFLTV FLAIPLAFIA GILFATLSCL HIWILMPFVK
TCLMVLPSVQ TIWKSVTDVI IAPLCTSVGR CFSSVSLQLS
QD
Cav2 polypeptide sequence

In view of the above known polypeptide sequences, probes and primers can be generated for use in the detection assays described above. Furthermore, known, commercially available primers and probe assays for the noted sequences are provided by Bio-Rad. Additionally improved antibodies can be developed using information gained from these initial products to improve target specificity.

### Examples

Experiments were designed to identify the levels of expression of ATP13A3 and/or c-myc correlate with polyamine transport. Cells with high levels c-myc have high basal levels of polyamine import and are sensitive to polyamine targeting therapies. In addition, cells with high basal levels of ATP13A3 readily escape DFMO only therapy by importing exogenous polyamines and require DFMO+PTI therapy in order to be effective in reducing tumor progression. Moreover, DFMO escape via exogenous polyamine import is especially facile in cells with high basal levels of ATP13A3 and low Cav-1 expression. These cell types (samples) which can escape DFMO-only therapy require DFMO+PTI therapy to effectively reduce tumor progression. Endpoints for this DFMO+PTI combination therapy (or for DFMO-only therapy if transport is not appreciably invoked by the DFMO treated cells) can include significantly altered intracellular polyamine levels (especially reduced putrescine and/or spermidine), S-adenosylmethionine (SAM), thymidine levels and CD-47. (See, David R. Soto-Pantoja, Erica V. Stein, Chengyu Liu, Abdel G. Elkahloun, Michael L. Pendrak, Alina Nicolae, Satya P. Singh, Zuqin Nie, David Levens, Jeffrey S. Isenberg & David D. Roberts. Thrombospondin-1 Signaling through CD47 Inhibits Self-renewal by Regulating c-Myc and Other Stem Cell Transcription Factors. Scientific Reports 2013, 3, 1673; doi:10.1038/srep01673). Cells with high levels of Cav-1 and low levels of Cav-2 are found in some of the most resistant cancer cells (i.e., melanomas), whereas cells with low Cav-1 and high Cav-2 levels are particularly sensitive to compounds A and B which are described in greater detail below. Compound A is a cytotoxic polyamine transport ligand that outcompetes native polyamines for cell entry (see FIGS. 16-19).

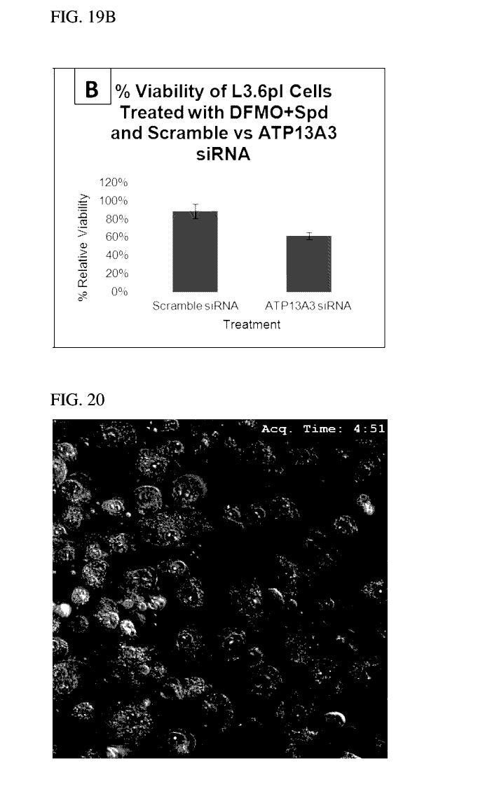

The inventors used Polyamine Transport Assays to show, for example, that cells like CHO, L3.6pl and PanO2 cells which have high polyamine transport activity also have high c-myc expression and are susceptible to therapies which target polyamine transport or metabolism. Additionally, cells which had high ATP13A3 expression were shown to readily import spermidine to escape DFMO treatment. Caveolin-1 by itself did not track well with basal polyamine uptake but low Cav-1 and high ATP13A3 were hallmarks of cells which readily imported spermidine to escape DFMO treatment. In some examples described below and shown in the FIGS, cells treated with compound A and/or compound B reduced in viability, whereas cells treated with compound A and varying quantities of exogenous Spermidine (Spd) had varying levels of viability indicating that compound A, competes with Spd for cell entry, for example. This confirms compound A is a polyamine transport targeting compound (see FIG. 19). Because they compete with native polyamines for cell entry, Compounds A and B are polyamine transport inhibitors which also have cytotoxic properties. We note that the trimer44NMe PTI is also a competitive inhibitor of polyamine uptake but is much less toxic than A and B. In this regard compounds can be grouped together as cytotoxic polyamine targeting probes (e.g., Ant44, compounds A and B) which outcompete native polyamines for cell entry and kill cells versus the relatively non-toxic polyamine transport inhibitors (like trimer44NMe or GW5074) which also outcompete native polyamines for cell entry but do not result in a cytotoxic response at the low doses seen with the cytotoxic probes.

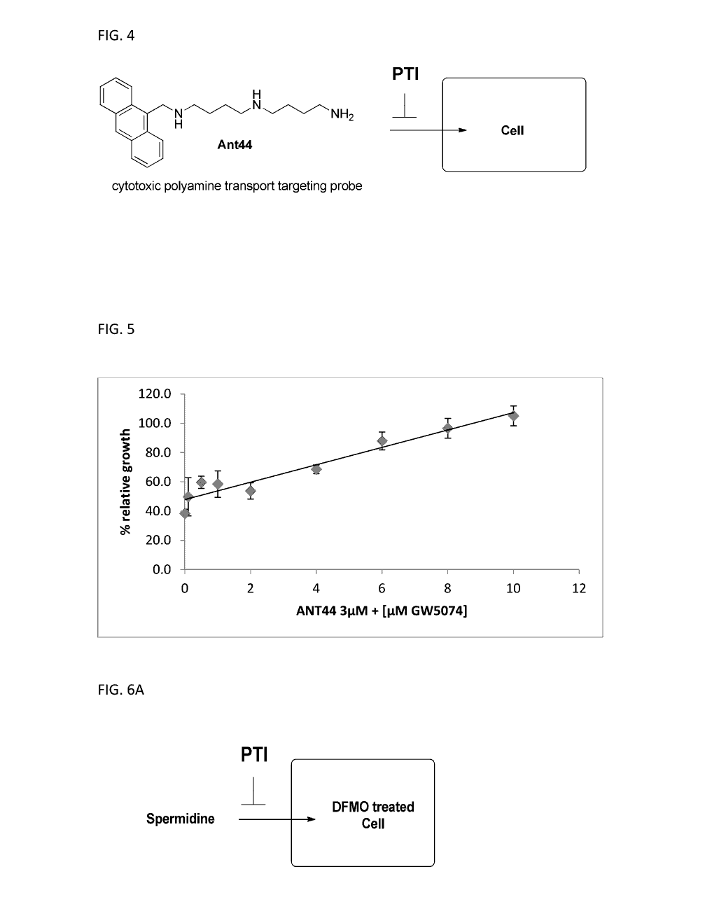

Polyamine transport inhibitor (PTI) Assay 1 was performed to block the uptake of a cytotoxic polyamine probe (Ant44) by a polyamine transport inhibitor (PTI) as shown in the diagram of FIG. 4 (Ant44 assay) (see Polyamine transport inhibitors: Design, Synthesis and Combination therapies with Difluoromethylornithine. Aaron Muth, Jennifer Archer, Nicolette Ocampo, Meenu Madan, Luis Rodriguez, and Otto Phanstiel IV. J. Med. Chem. 2014, 57, 348-363).

A successful PTI will block the entry of the cytotoxic Ant44 probe into cells and result in significantly enhanced cell viability versus the Ant44 treated control. The inventors have found that a non toxic PTI rescues cells from the toxic Ant44 probe. Cells that do not have active transport will be much less sensitive to the Ant44 probe, i.e., CHO vs CHO-MG cells. See “N¹-Substituent Effects in the Selective Delivery of Polyamine-Conjugates into Cells Containing Active Polyamine Transporters” Gardner, R. A.; Delcros, J-G.; Konate, F.*; Breitbeil III, F.; Martin, B.; Sigman, M.; Huang, M.; Phanstiel IV, O. J. Med. Chem. 2004, 47, 6055-6069. Using this assay 1 seen in FIG. 4, the inventors showed that CHO K1 cells can be rescued from Ant44 by the addition of a PTI (GW5074, Drug 6).

The results of the assay 1 can be seen in the table and graph of FIGS. 5A-B which show CHO cells are protected from a cytotoxic dose of the polyamine transport ligand Ant44 (3 μM) in the presence of a polyamine transport inhibitor (PTI) GW5074. As shown in the graph CHO cells dosed with Ant44 (3 μM) gave 38% viability. Cell viability was increased in a dose dependent manner when the Ant44 concentration was held fixed at 3 μM and the GW5074 PTI concentration was increased from 0 to 10 μM (as shown in the x-axis). The GW5074 PTI compound was not toxic to CHO cells at 10 μM. A similar rescue from Ant44 was noted for the addition of exogenous spermidine and suggests that the Ant44 compound uses the polyamine transport system for cell entry.

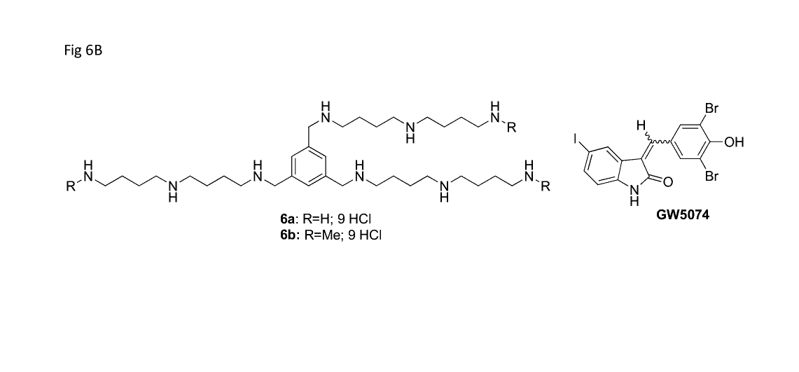

FIG. 6A shows a polyamine transport inhibitor assay 2, wherein the uptake of a rescuing dose of spermidine into DFMO-treated cells is blocked by the PTI. DFMO was dosed to give 50% relative viability versus an untreated control, i.e. DFMO is dosed at its IC50 value. Exogenous spermidine (Spd, 1 μM) is added to the DFMO treated cells and provided significant rescue of these cells to >80% viability depending upon the cell line. A successful PTI was able to block the entry of the spermidine rescuing agent into DFMO treated cells and result in a cell viability near the DFMO control (i.e. 50% viability). The inventors identified the following cell viabilities: DFMO only (50%), Spd only (100%), PTI (>90%), DFMO+Spd (>80%), DFMO+Spd+PTI (50%). The PTI provides a dose dependent lowering of cell viability in the presence of DFMO and a rescuing dose of spermidine. Since this assay resulted in reduced cell viability it is important not to use the PTI at a concentration which imparts significant toxicity to the cell. For this reason, the inventors did not use the PTI above its IC₁₀ value. Identifying cells that respond to exogenous spermidine allow this assay to be possible and the c-myc and ATP13A3 biomarkers help predict these outcomes as well as the Vmax measurements for ³H Spd import.

Assay 2 was used to screen the GW5074 PTI in L3.6pl cells which has high expression of c-myc and ATP13A3. Using L3.6 pl cells, 500 cells/well, incubated for 48 h at 37° C. along with 250 μM aminoguanidine (AG, to inhibit the amine oxidases in the fetal bovine serum which could degrade the spermidine added), the inventors showed that DFMO alone and in combination with the GW5074 polyamine transport inhibitor is effective against L3.6pl cells containing high expression of the two biomarkers.

FIG. 6B details the structures of validated PTIs, Compounds 6a and 6b are polyamine based PTIs and GW5074 is a non-polyamine based PTI which is also a c-Raf inhibitor.

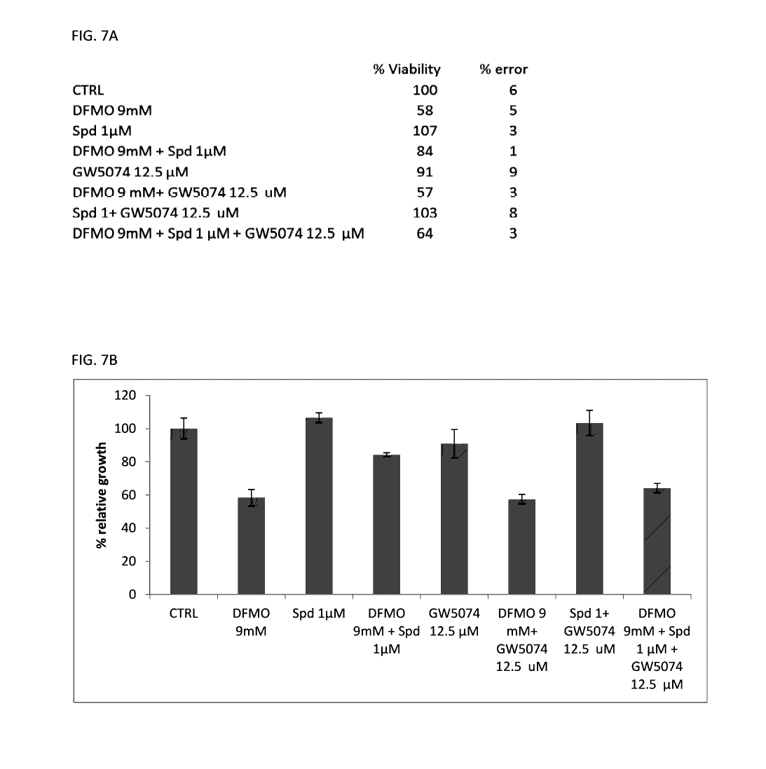

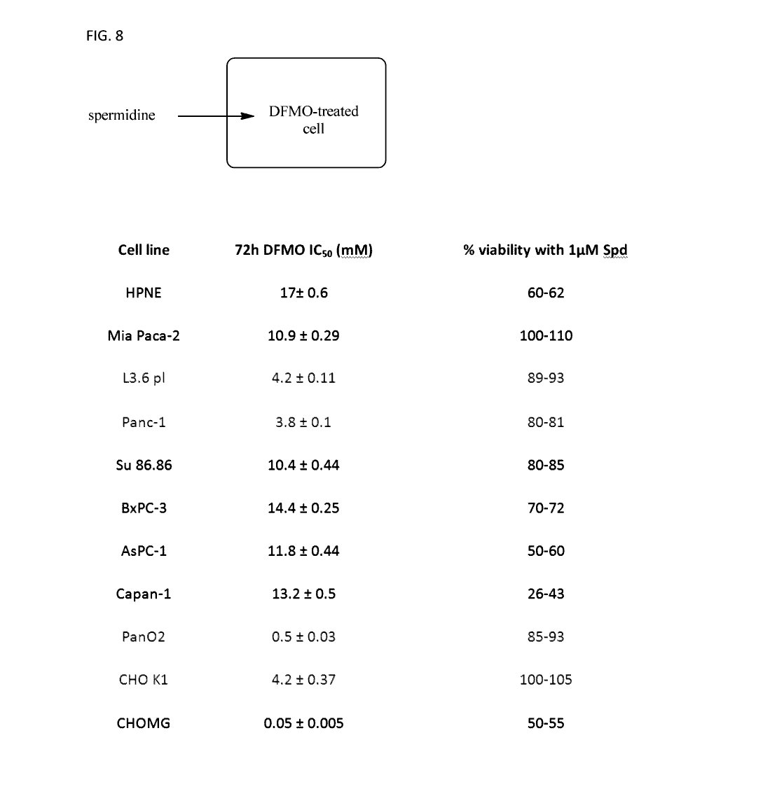

FIGS. 7A-B provide a table and graphically illustrated results showing a combination therapy of DFMO+PTI (GW5074) in the presence and absence of exogenous spermidine. The inventors also determined the sensitivity of cells to DFMO and their ability to be rescued by exogenous spermidine (shown in the table of FIG. 8). It is provided herein that cells that are committed to polyamine transport for their polyamine supplies are sensitive to DFMO (DFMO IC₅₀<5 mM) due to their reduced reliance upon polyamine biosynthesis and may be readily rescued by exogenous spermidine (Spd) due to their high import rates to viability levels >80%. L3.6pl, Panc1, CHO and PanO2 cells all had this property and are highlighted in red in FIG. 8.

FIG. 8 provides a table illustrating that cells had differential sensitivity to DFMO and some were not rescue-able from DFMO even with exogenous spermidine addition (AsPC-1 and Capan-1). The inventors later identified that these two non-responding cell lines have virtually no detectable ATP13A3 expression and very low c-myc expression, whereas the other responders expressed both of these key markers of polyamine import. The inventors concluded that cells which are c-myc driven have a high reliance upon polyamine import and would be sensitive to the DFMO+PTI combination therapy. The L3.6pl cell line which had high c-myc expression also had the highest Vₘₐₓ and was one of the most sensitive to DFMO. This result was attributed to the cell's commitment to polyamine import. ATP13A3 indicates the ability of the cell to import spermidine in the presence of DFMO. As such the rescue-ability of the cell lines was predicted by their relative basal expression of ATP13A3 (without DFMO addition). When DFMO inhibits the ability of the L3.6pl cells to make polyamines via ornithine decarboxylase, the cells upregulate their polyamine transport system (PTS) to recover. The inventors have shown herein in FIG. 9A that the expression of ATP13A3 was also shown to increase in the presence of DFMO treatment in the responding cell lines, i.e. cell lines that could be rescued from DFMO by exogenous spermidine. If there is no exogenous spermidine, then the cells are in crisis and succumb to the effects of DFMO and result in a low DFMO IC₅₀ value.

ATP13A3 is the human analogue of CATP-5, a gene shown to be involved in polyamine transport in C. elegans (see Caenorhabditis elegans P_{5B}-type ATPase CATP-5 operates in polyamine transport and is crucial for norspermidine-mediated suppression of RNA interference. Heinick, A. Urban, K.; Roth, S.; Spies, D.; Nunes, F.; Phanstiel IV, O.; Liebau, E. Lüersen, K. FASEB Journal 2010, 24, 206-217.)

The results provided herein supports the finding that ATP13A3 plays an important role in human polyamine transport because only those cell lines which had significant expression of this marker were shown to be rescued by exogenous spermidine indicating this marker as a biomarker of DFMO escape via polyamine import. This marker was also shown to be significantly upregulated in L3.6pl cells in the presence of DFMO (72 h incubation) and modulated (lowered) by exogenous spermidine levels in the presence of DFMO (FIG. 9).

FIGS. 9A-B provide graphical illustrations of the modulation of ATP13A3 and Cav-1, respectively, by DFMO and DFMO+Spermidine in L3.6pl human pancreatic cancer cells after 72 h incubation at 37° C. In FIG. 9A ATP13A3 protein levels after 72 h incubation at 37° C. are shown in L3.6pl (top) and Bx-PC3 (bottom) pancreatic cancer cells in the presence of DFMO or DFMO+Spd as analyzed by Western blot. Expression was normalized using β-actin levels. Both cell lines showed a significant increase in relative ATP13A3 expression in the presence of the 48 h IC₅₀ DFMO dose (e.g., 8 mM) or DFMO+(1 μM Spermidine). In FIG. 9B (top and bottom), the levels of Cav-1 after 72 h incubation at 37° C. were not dramatically changed in either L3.6pl or BxPC-3 pancreatic cancer cells in the presence of DFMO indicating that the basal Cav-1 levels do not change dramatically with DFMO addition. Note an increase in Cav-1 levels would stabilize caveolae at the cell surface and reduce the uptake of polyamines via a caveolin dependent pathway. A decrease in Cav-1 levels would increase polyamine import.

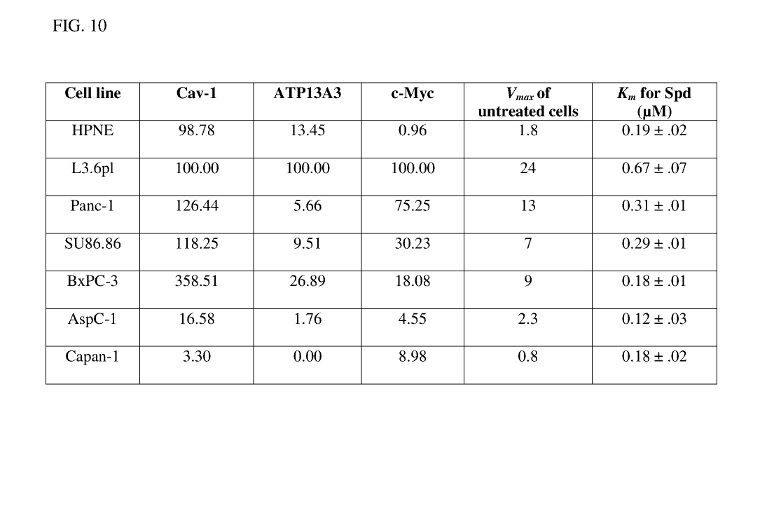

FIG. 10 provides a table showing the relative protein expression of Cav-1, ATP13A3 and c-myc in human pancreatic cancer cell lines (normalized by dividing by the relative beta actin expression level) along with the respective Vₘₐₓ values for ³H Spd import and Km values. The entries represent 100%×protein expression/beta actin expression is shown in the table along with the Vₘₐₓ and Km values reported earlier. As mentioned earlier, a strong correlation was found between ATP13A3 expression and the ability of DFMO-treated cells to be rescued by exogenous spermidine (FIG. 8, rightmost column).

The results identified in the in vitro studies provide that high c-myc and ATP13A3 expression levels were good indications of cell lines that had high polyamine import activity (high Vₘₐₓ) and were rescue-able by exogenous spermidine (in the presence of DFMO). These cells were still sensitive to blockade of polyamine import but required higher doses of PTI to be effective. PTIs 6a and 6b (FIG. 6B) were effective PTI agents in vitro. The N-methylated PTI 6b was chosen for in vivo studies due to its enhanced stability towards amine oxidases. L3.6pl cells had the highest Vₘₐₓ and good sensitivity to DFMO and was therefore chosen for in vivo evaluation and for its demonstrated in vitro sensitivity to the combination therapy of DFMO+PTI.

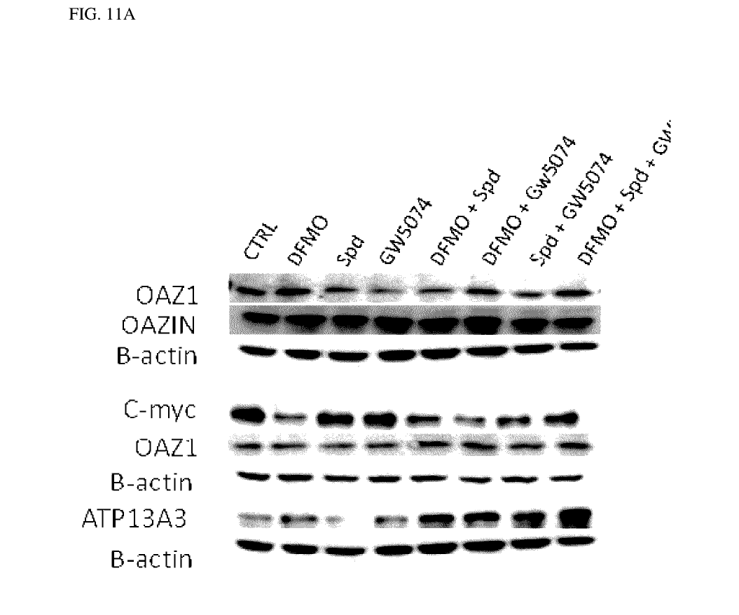

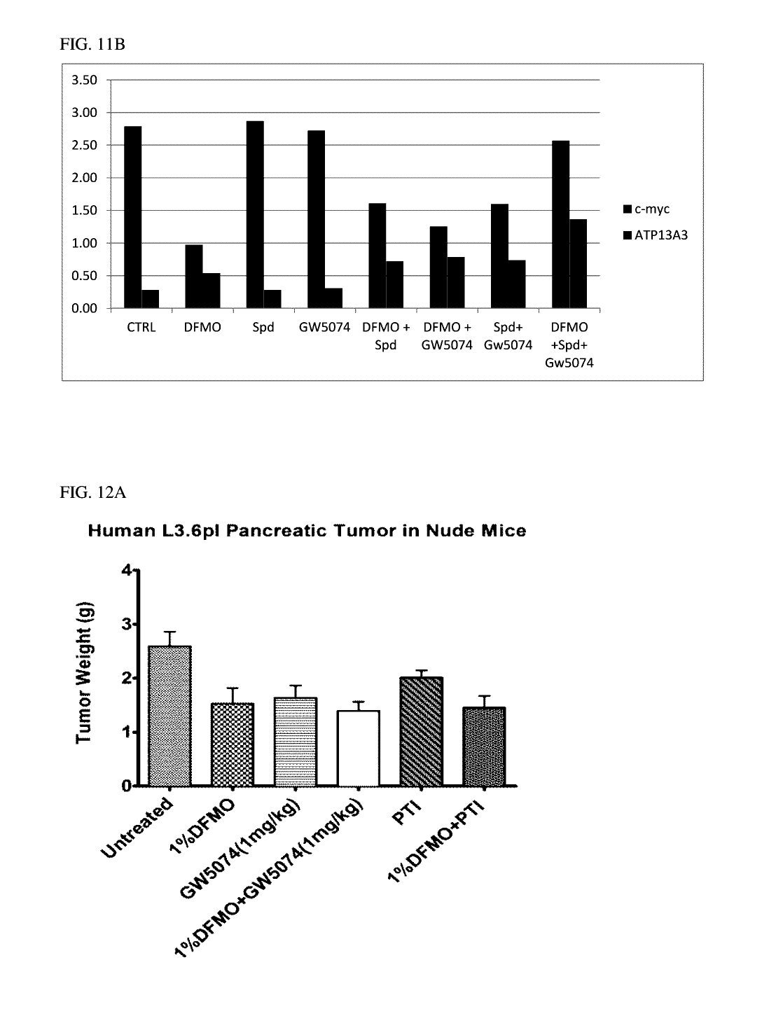

FIG. 11A-B demonstrate modulation of c-Myc and ATP13A3 protein expression in human L3.6pl cells (normalized vs beta-actin control) in the presence of a PTI (GW5074), DFMO and Spermidine. The results shown provide that c-Myc and ATP131A3 serve as biomarkers for polyamine transport activity and can be used to identify tumors or cell lines that may be most susceptible to polyamine targeting therapies (like DFMO+PTI combination therapy or aryl-polyamine conjugates which enter and kill cells via their active polyamine transport system). These results show that the therapies are modulating c-myc and ATP13A3 levels in these cancer cells. Note: antizyme 1 (OAZ1) and antizyme inhibitor (OAZIN) were also measured.

### Animal Experiments

These experiments demonstrate that cell lines like L3.6pl and PanO2 with high polyamine transport and high expression of c-myc and ATP13A3 are sensitive to polyamine targeting therapies in vitro. The extension of these findings is that cells which have high c-myc and ATP13A3 expression will be sensitive to the therapy described herein. These therapies were evaluated in vivo. The PTI, GW5074 compound (MW=520 g/mol), was used at 1 mg/kg and compared with the polyamine based PTI 6b at an equimolar dose (1.8 mg/kg; MW=962.19 g/mol) in three animal studies.

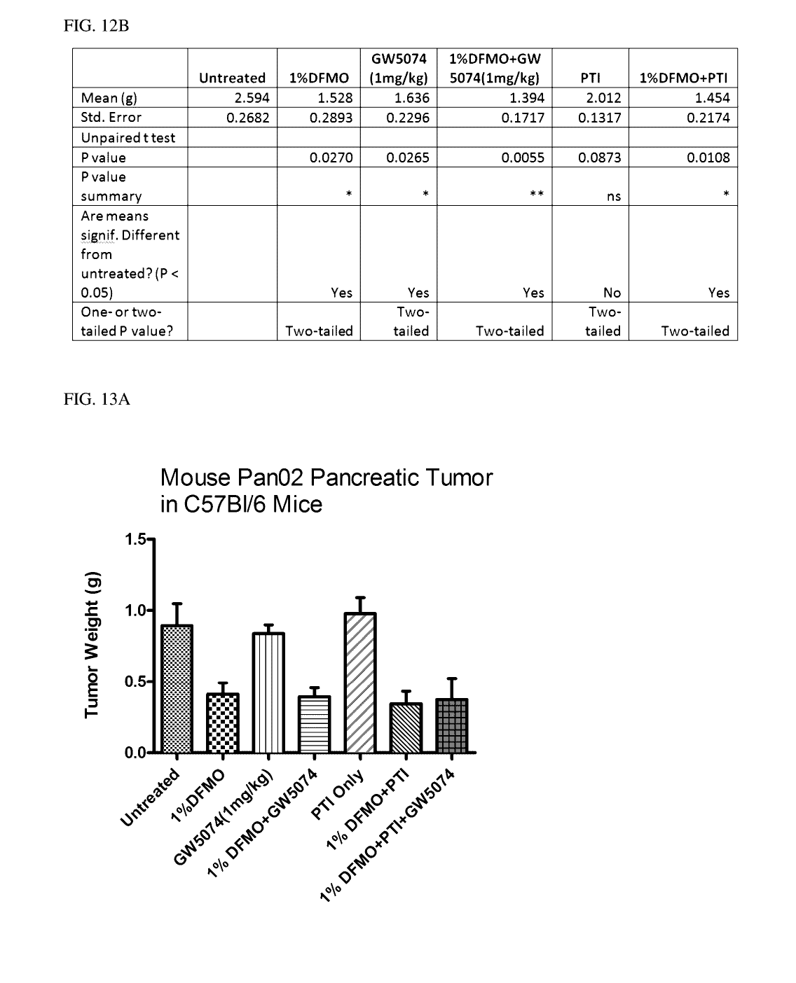

The first set of experiments was in nude mice (immune compromised) using human L3.6pl pancreatic cancer cells which have high ATP13A3 and high c-myc expression and high Vₘₐₓ. The An experiment was conducted to look at combination therapies of the GW5074 compound with DFMO (an inhibitor of ODC), FIG. 12A-B. The polyamine-containing trimer44NMe (6b) PTI (FIGS. 6B and 12) was also used for comparison. Note TTI′ in FIGS. 12 and 13 refer to the polyamine-based trimer44NMe PTI compound.

FIGS. 12A-B provide a graphical illustration and a table with results showing GW5074 has promise in inhibiting in vivo pancreatic tumor growth in combination with DFMO. 0.5×10⁶ L3.6pl tumor cells were injected into the pancreas of nude mice and allowed to grow for 1 week. 1% DFMO was administered in the drinking water (changed weekly) for 2 weeks. GW5074 at the lower dose of 1 mg/kg that could synergize with 1% DFMO was injected intraperitoneally (i.p.) for 5 days per week for 2 weeks. The same regimen was implemented for the PTI trimer44NMe (PTI) at equimolar concentration to the GW5074. Mice were collected and pancreatic tissue/tumor, with spleen attached, was weighed. The table in FIG. 12B shows the data of the results provided as graphically illustrated in FIG. 12A. FIG. 12B shows a table representing human L3.6pl Pancreatic Tumor in nude mice-combination with 1% DFMO and GW5074, and PTI in varying combinations as shown.

An additional animal study used immune competent C57Bl/6 mice and the PanO2 mouse pancreatic cancer cell line, (FIG. 13A-B). The GW5074 and trimer44NMe PTI were compared for their ability to reduce tumor size alone or in combination with DFMO. FIGS. 13A-B include a graphical illustration and a table showing PanO2 in vivo pancreatic tumor growth was inhibited in combination with DFMO. 1×10⁶ PanO2 tumor cells were injected into the pancreas of C57Bl/6 mice and allowed to grow for 2 weeks. 1% DFMO was administered in the drinking water (changed weekly) for 2 weeks. GW5074 at the lower dose of 1 mg/kg that could synergize with 1% DFMO was i.p. injected for 5 days per week for 2 weeks. The same regimen was implemented for the PTI trimer44NMe (PTI) at equimolar concentration to the GW5074. Mice were collected and pancreatic tissue/tumor, with spleen attached, was weighed.

FIG. 13B is a table providing results of the study of Mouse PanO2 Pancreatic Tumor in C57Bl/6 Mice-Combination with 1% DFMO shown in the graphical illustration of FIG. 13A. These results show that all DFMO containing experiments gave a statistical reduction in tumor size. The inventors have demonstrated that the PTI agents are well tolerated in mice and give a statistically significant reduction in tumor size. The GW5074 compound by itself gave a statistically significant reduction in tumor for the nude mouse experiment with human L3.6pl cells but not with murine PanO2 cells in immune competent C57Bl/6 mice. Each combination therapy (DFMO+trimer44NMe and DFMO+GW5074) when tried in both experimental models (nudes and C57Bl/6 mice) gave statistically significant reductions in tumor size (see FIGS. 12B and 13B).

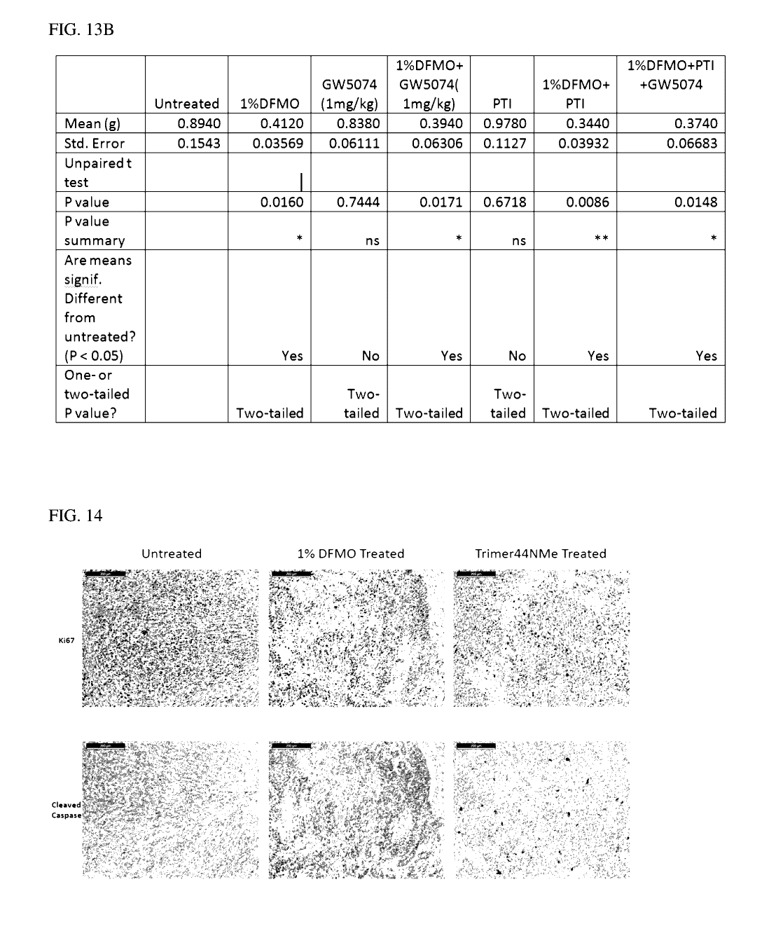

FIG. 14 shows a histology of PanO2 derived tumors in C57Bl/6 mice. Ki67, a proliferation marker and cleaved caspase were imaged via histological stains and showed a reduced proliferation in the DFMO and Trimer44NMe (Compound 6b) panels. The trimer44NMe panel (bottom right panel) showed dark staining in specific areas for the cleaved caspase indicating that apoptosis is occurring in tumors treated with the PTI (trimer44NMe) (Polyamine transport inhibitors: Design, Synthesis and Combination therapies with Difluoromethylornithine. Aaron Muth, Jennifer Archer, Nicolette Ocampo, Meenu Madan, Luis Rodriguez, and Otto Phanstiel IV. J. Med. Chem. 2014, 57, 348-363) by itself.

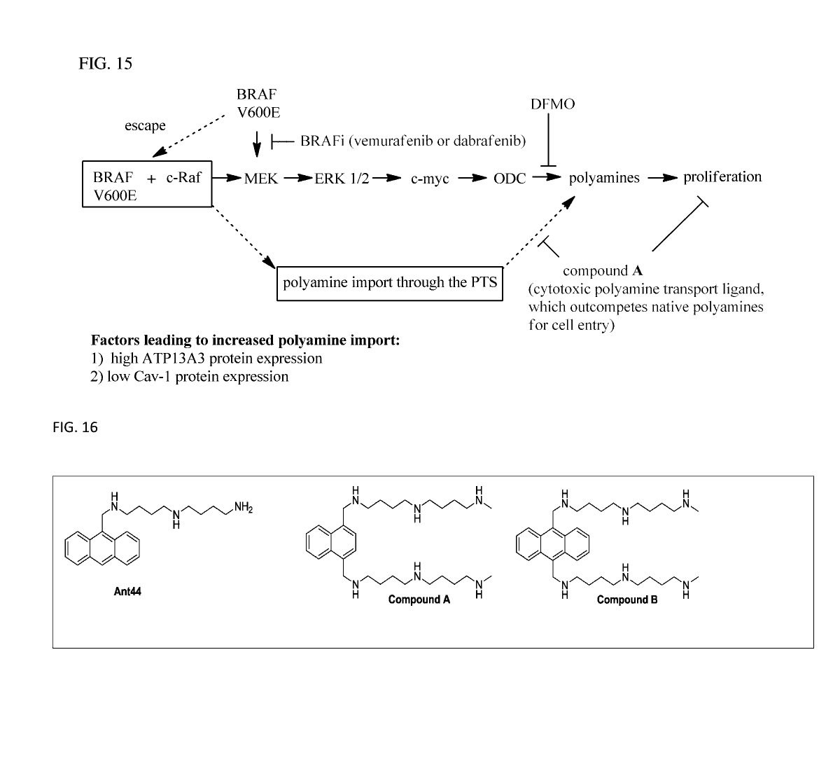

FIG. 15 shows a signaling pathway for melanoma BRAF and polyamine transport targeting compounds, e.g., compound A to inhibit polyamine transport in the signaling pathway. Compound A, for example, may inhibit polyamine import into cells via the polyamine transport system (PTS) by, for example outcompeting native polyamines for cell entry. Compound A may also inhibit proliferation of polyamines as provided in the diagram. Compound A is a polyamine transport inhibiting compound with cytotoxic properties. In FIG. 16, the molecular structure for polyamine transport selective probes Ant44, compound A, and compound B are provided.

FIG. 17A shows a Western blot analysis of Caveolin proteins in melanoma cell lines showing high levels of Cav-1 and low levels of Cav-2 in the most resistant melanoma cell line (LOX IMVI) to compound A. Eustace, A. J.; Kennedy, S.; Larkin, A. M.; Mahgoub, T.; Tryfonopoulos, D.; O'Driscoll, L.; Clynes, M.; Crown, J.; O'Donovan, N. Predictive biomarkers for dasatinib treatment in melanoma. Oncoscience 2014, 1 (2), 158-166. Decreased viability of cells when treated with compound A was most significant in the MALME-3M cells (see table of FIG. 18A). These MALME-3M cells contain higher levels of Cav-2 and lower levels of Cav-1.

FIG. 17B shows selective targeting of melanomas of various cell types and cell sensitivity to compound A (10 μM) is shown in the NCI 60-cancer cell screen. Graphical bars extending to the right of the 0 growth percent line indicates growth inhibition by compound A of those specific cell lines. MALME-3M was the most sensitive cell line to compound A as shown, which includes low Cav-1 and high Cav-2 levels. Compound A gave a 96 h IC₅₀ of 14 nM in MALME-3M. FIG. 17B also provides evidence of decreased growth of cancer cell lines (i.e., MALME-3M, M14, MDA-MB-435, SK-MEL2, SK-MEL-28, SK-MEL-5, UACC-257, and UACC-62) when exposed to compound A.

FIG. 17C shows a graphical illustration of selective targeting of melanomas and cell sensitivity to anthracenyl derivative B (10 μM) in the NCI 60-cancer cell screen. As in FIG. 17B, a bar extending to the right of the 0 growth (central line) in FIG. 17C indicates growth inhibition by compound B. MALME-3M (low Cav-1 and high Cav-2) was the most sensitive cell line to compound B. Compound B gave a 96 h IC₅₀ of 62 nM in MALME-3M and was used in confocal microscopy studies showing rapid cell uptake and nuclear import in cultured MALME-3M cells (see FIG. 20).

FIG. 18 provides a graph of cells incubated for 96 h at 37 degrees Celsius with compound A at 0.02 μM (MALME-3M cells) and 0.8 μM (MALME-3 control cells) demonstrating the percent viability of the cells. Aminoguanidine (AG) at 1 mM was determined to be non-toxic and was incubated with cells for 24 h prior to compound addition. Control represents the untreated cells. All experiments were performed in triplicate. Spermidine (Spd, 100 μM) was non-toxic to both cell lines. As provided in the figure, compound A competes with Spd for cell entry. Compound A is a valid polyamine transport probe.

FIG. 20A provides a graphical illustration of ATP13A3 protein levels in L3.6pl cells as analyzed by Western blot. Expression was normalized by dividing by β-actin protein levels. Decreased ATP13A3 protein expression was observed in the presence of the ATP13A3 siRNA vs the scrambled siRNA (p<0.05; each at 75 nM).

FIG. 20B provides a graphical illustration of the effect of scrambled siRNA vs. ATP13A3 siRNA on relative L3.6pl cell viability % when challenged with a 48 h IC50 dose of DFMO (8 mM) and a rescuing dose of Spd (1 μM). The data provided shows reduced ATP13A3 expression and associated reduced rescue by Spd (p, 0.01) by the ATP13A3 siRNA. This demonstrates the involvement of ATP13A3 in polyamine transport.

FIG. 21 is a confocal microscopy view of MALME-3M cells incubated with the PTS-selective compound B (2.5 μM). The MALME-3M cells were mounted on microscope slides and were placed into a Tokai Hit Chamber to control humidity, temperature and CO₂ levels over the course of the experiment. A NIKON A1 Laser scanning confocal microscope equipped with a 405 nm excitation laser light and a transmitted light detector was used to acquire images over time. The image was acquired at 4 h 51 min after addition of B. The time-course study showed a dose dependent uptake of compound B over time. Compound B, which appears blue under the 405 nm laser excitation, becomes very apparent at the 5 hour time point inside the nuclear compartment. This demonstrates cellular uptake of this molecule and a time-course preferred localization to the nucleus.

Table 1 compares unmethylated Compound A (containing primary amine termini), and N-methylated Compound A (containing N-methyl termini). Methylated Compound A (having terminal N-methyl groups) was found to be metabolically stable to amine oxidases. Due to its metabolic stability, methylated Compound A was found to outlast unmethylated Compound A, particularly in the presence of amine oxidases, in a non-limiting embodiment. As shown in Table 1 below, without the protective aminoguanidine (AG) amine oxidase inhibitor, unmethylated Compound A molecules were less selective and less metabolically stable when compared to the sample containing AG (used to inhibit the degradative action of amine oxidases on the primary amine present in the unmethylated compound A). However, N-methylated Compound A was highly selective in targeting CHO cells with active PTS (ratio >2200). This is due to the presence of the terminal N-methyl groups in A.

Table 2 below provides a comparison of methylated vs. unmethylated Compound A in selective targeting of melanoma cells.

TABLE 1
PTS targeting by Compound A using PTS-active CHO and PTS-deficient CHO-MG*
cells^{a}

| | | | | CHO- | | |
|---|---|---|---|---|---|---|
| | CHO- | CHO | | MG* | CHO | IC₅₀ |
| | MG* IC₅₀ | IC₅₀ | IC₅₀ | IC₅₀ | IC₅₀ | ratio^{c} |
| | (μM) | (μM) | ratio^{c} w/ | (μM) | (μM) | w/o |
| Compound | w/ AG | w/ AG | AG | w/o AG | w/o AG | AG |
| Compound A | >100 | 0.022 | >4545 | 52.1 | 5.5 | 9.5 |
| w/o Me | | (±0.002) | | (±7.5) | (±0.5) | |
| (H₂N44Nap44NH₂) | | | | | | |
| Compound A | >100 | 0.044 | >2272 | >100 | 0.039 | >2564 |
| (MeHN44Nap44NHMe) | | (±0.002) | | | (±0.001) | |

^{a}A high CHO-MG*/CHO IC₅₀ ratio denotes high selectivity in killing cells with an active polyamine transport system, PTS. Aminoguanidine (AG) is added to cell culture to inhibit the action of amine oxidases found in calf serum. When this protective AG agent is removed, compound A retains its high selectivity in targeting CHO cells with active PTS (ratio >2200). This is due to the presence of the terminal N-methyl groups in A. In contrast when these N—Me groups are not present (top entry) and AG is not added, there is a dramatic loss in selectivity (IC₅₀ ratio changes from >4545 to 9.5). Thus, we have designed around this issue via N—Me groups.

TABLE 2
Selective Targeting of metastatic melanoma cells
(MALME-3M) over its matched control (MALME-3)^{b}

| | MALME-3 | MALME-3M | |
|---|---|---|---|
| | IC₅₀ | IC₅₀ | IC₅₀ |
| Compound | (μM) | (μM) | ratio^{c} |
| Compound A w/o Me | 1.21 | 0.018 | 71 |
| (H₂N44Nap44NH₂) | (±0.09) | (±0.001) | |
| Compound A | 0.82 | 0.014 | 59 |
| (MeN44Nap44NMe) | (±0.06) | (±0.001) | |

^{b}a high IC₅₀ ratio of MALME-3/MALME-3M denotes selective targeting of melanoma cells. Both compound demonstrate selective killing of melanomas in the presence of aminoguanidine (1 mM), which was added to allow the comparison as the top entry is unstable without AG present (See Table 1). Compound A (which has N-methyl groups) provides specific targeting ability and enhanced stability which obviates the need to add an amine oxidase inhibitor.

Table 3 shows a comparison of the different protein markers and the Vmax and degree of Spermidine rescue in a side by side comparison.

TABLE 3
Relative Protein expression of Cav-1, ATP13A3 and c-Myc
in untreated human pancreatic cancer cell lines along with the respective
Vₘₐₓ values for ³H Spd import and relative spermidine (Spd)
rescue in DFMO-treated cells index.^{c}

| | | | | Vₘₐₓ of ³H | |
|---|---|---|---|---|---|
| | | | | Spd | Spd |
| | | | | untreated | rescue |
| Cell line | Cav-1 | ATP13A3 | c-Myc | cells | index |
| HPNE | 98.78 | 13.45 | 0.96 | 1.8 | very low |
| L3.6pl | 100.00 | 100.00 | 100.00 | 24 | high |
| Panc-1 | 126.44 | 5.66 | 75.25 | 13 | med |
| SU86.86 | 118.25 | 9.51 | 30.23 | 7.0 | med |
| BxPC-3 | 358.51 | 26.89 | 18.08 | 9.0 | low |
| AspC-1 | 16.58 | 1.76 | 4.55 | 2.3 | Not rescuable |
| Capan-1 | 3.30 | 0.00 | 8.98 | 0.8 | Not rescuable |

^{c}Entries represent protein expression (normalized by beta-actin expression) and expressed in relative %, where the expression level of each protein found in L3.6pl cells was set to 100%. To illustrate how the relative protein expression patterns relate to the polyamine transport properties of the human cell lines, the Vₘₐₓ value for ³H-Spd import (nmoles Spd/mg protein/min) and the Spd rescue index observed with DFMO-treated cells are re-listed for comparisons. Cell lines with high ATP13A3 and high c-myc had high Vmax and high Spd rescue outcomes, which indicates that these cell types will circumvent DFMO via increased polyamine import and would be good candidates to be treated with DFMO + PTI therapy.

In embodiments provided herein, a diagnostic and therapeutic treatment method for treating cancer cells in a patient are provided including, determining a level of Cav-1 and a level of Cav-2 in a cell sample from the patient, comparing the levels of Cav-1 and Cav-2 in the cell sample to the levels of Cav-1 and Cav-2 a control sample or to standard values of Cav-1 and Cav-2, wherein a lower level of Cav-1 and a higher level of Cav-2 in the cell sample relative to the control sample or to the standard values is indicative of cancer cells that are susceptible to treatment with a polyamine transport inhibitor (PTI), and treating the cancer cells with the PTI, DFMO, or a combination thereof.

In one non-limiting embodiment, the method is provided wherein the cancer cells are melanoma cells. In a further non-limiting embodiment, the melanoma cells are MALME-3M cells. In other non-limiting embodiments, the melanoma cells include: M14, MDA-MB-435, SK-MEL-2, SK-MEL-28, SK-MEL-5, UACC-257, or UACC-62 cells.

In one particular non-limiting embodiment, the method may be provided wherein the PTI includes Compound A, Compound B, or a combination thereof.

In another embodiment, a method of detecting cancer cells susceptible to treatment with a polyamine transport inhibitor (PTI) in a patient is provided. The method includes determining a level of ATP13A3 and a level of Cav-1 in a cell sample from the patient and comparing the levels of ATP13A3 and Cav-1 in the cell sample to the levels of ATP13A3 and Cav-1 in a control sample or standard values of ATP13A3 and Cav-1, wherein higher level of ATP13A3 and a lower level of Cav-1 in the sample relative to the control sample or standard values is indicative of cancer cells that are susceptible to treatment with a PTI. The method may further include determining a level of Cav-2 in the cell sample and comparing the level of Cav-2 in the cell sample to the level of Cav-2 in a control sample or to a standard value of Cav-1, wherein a higher level of Cav-2 in the cell sample relative to the control sample or the standard value for Cav-1 is indicative of cancer cells in the patient that are susceptible to treatment with a PTI. The method may further include treating the cancer cells with a polyamine biosynthesis inhibitor. In one non-limiting embodiment, the polyamine biosynthesis inhibitor comprises DFMO (difluoromethylornithine).

In other embodiments, a method for detecting cancer cells susceptible to treatment with a polyamine transport inhibitor (PTI) is provided. The method includes receiving a cell sample from a patient, presenting a first reagent and a second reagent to the cell sample to detect differential levels of Cav-1 and Cav-2, or nucleic acid sequences encoding the same, in the cell sample, wherein the first reagent is specific to Cav-1, the second reagent is specific to Cav-2, under conditions to detect binding of the first and second reagents to Cav-1 and Cav-2, respectively, or hybridization of the first and second reagents to the nucleic acid sequences, and determining that cancer cells in the patient are treatable with a polyamine transport inhibitor if a higher level of Cav-2 and a lower level of Cav-1, or nucleic acid sequences encoding same, than in normal cells was determined to be present in the cell sample.

Another embodiment includes a method of treating cancer cells susceptible to treatment with a polyamine transport inhibitor (PTI). The method includes receiving a cell sample from a patient, presenting a first reagent and a second reagent to the cell sample to detect differential levels of Cav-1 and Cav-2, or nucleic acid sequences encoding the same, in the cell sample, wherein the first reagent is specific to Cav-1, the second reagent is specific to Cav-2, under conditions to detect binding of the first and second reagents to Cav-1 and Cav-2, respectively, or hybridization of the first and second reagents to the nucleic acid sequences, determining that cancer cells in the patient are treatable with a polyamine transport inhibitor if a higher level of Cav-2 and a lower level of Cav-1, or nucleic acid sequences encoding same, than in normal cells was determined to be present in the cell sample, exposing the cancer cells to the polyamine transport inhibitor to cause a reduction in viability of the cancer cells.

In still another embodiment, an ELISA microarray testing kit for detecting cancer cells susceptible to treatment with a polyamine transport inhibitor (PTI) is provided. The testing kit may include a component comprising a substrate to which ATP13A3 and Cav-1 in a cell sample can bind, wherein the substrate comprises an array of individual antibody loci comprising antibodies specific to ATP13A3 and/or Cav-1, one or more reagents configured to detect ATP13A3 and Cav-1 bound to the substrate, wherein the detection of a high level of ATP13A3 and a low level of Cav-1 in the cell sample when compared to a control is indicative of cancer cells susceptible to treatment with a polyamine transport inhibitor. The ELISA microarray testing kit may further include a substrate to which c-myc in the cell sample can bind, wherein the substrate comprises individual antibody loci comprising antibodies specific to c-myc, the microarray testing kit further including one or more reagents configured to detect c-myc bound to the substrate, wherein the detection of a high level of c-myc in the cell sample when compared to a control is indicative of cancer cells susceptible to treatment with a polyamine transport inhibitor.

The ELISA microarray testing kit may further include a substrate to which Cav-2 in the cell sample can bind, wherein the substrate comprises individual antibody loci comprising antibodies specific to Cav-2, the microarray testing kit further including one or more reagents configured to detect c-myc bound to the substrate, wherein the detection of a high level of Cav-2 in the cell sample when compared to a control is indicative of cancer cells susceptible to treatment with a polyamine transport inhibitor, in a non-limiting example.

Primary antibodies covalently attached to respective fluorophores can be used to directly measure the relative expression levels of these biomarkers in histological samples either separately or together to create a mosaic readout of differential protein expression of the key biomarkers. Accordingly, in another embodiment, specific antibodies for ATP13A3, c-myc, cav-1 and Cav-2 are either covalently linked to different fluorophores (different colors) or detected by differently colored fluorescent secondary antibodies could be added to a histological sample and show a colored panel readout of the relative expression of each protein by immunocytochemistry, for example. These methods are further described in Controls for Immunocytochemistry: An Update, Burry, Richard W., Journal of Histochemistry & Cytochemistry 59(1) 6-12, 2011, which is incorporated by reference herein. This would provide a snapshot of the relative protein expression patterns of each of the biomarkers in the tissue sample. Another related embodiment pertains to a method that involves subjecting a tissue sample to a primary antibody_{A} that detects ATP13A3, a primary antibody_{B} that detects c-myc, a primary antibody_{C} that detects Cav-1, and a primary antibody_{D} that detects Cav-2 in a tissue sample, wherein upon subjecting the tissue sample to the solution, the primary antibodies_{A-D} bind specifically to proteins ATP13A3, c-myc, Cav-1, and Cav-2, respectively, in the sample, wherein the primary antibodies_{A-D} are each individually linked to different colored fluorophores Upon subjecting the tissue sample to the antibodies, the relative levels of the noted proteins can be visualized to provide a color mosaic that will indicate whether the tissue sample includes cells having an active PTA (polyamine transport activity).

Various polyamine biosynthesis inhibitors and PTI's are described herein, including, but not limited to, DFMO, N¹-(9-anthrylmethyl)-homospermidine (Ant44), MeN44Nap44NMe or GW5074. DFMO is a polyamine biosynthesis inhibitor. Polyamine transport inhibitors (PTI's) include, for example, compounds which inhibit the transport of polyamines into the cell, and compounds having cytotoxic properties which inhibit transport of polyamines into the cells, such as Compound A as described herein, in a non-limiting example. Polyamine transport inhibitors therefore include Compound A, Compound B in addition to other PTI's which may be used to inhibit polyamine transport in cells identified by methods described herein as being susceptible to polyamine transport inhibition. Additional PTI's which may be used can be found in the following: Polyamine transport inhibitors: Design, Synthesis and Combination therapies with Difluoromethylornithine. Aaron Muth, Jennifer Archer, Nicolette Ocampo, Meenu Madan, Luis Rodriguez, and Otto Phanstiel IV. J. Med. Chem. 2014, 57, 348-363; “Fluorescent Cytotoxic Compounds Specific for the Cellular Polyamine Transport System.” U.S. Pat. No. 8,410,311 (Apr. 2, 2013); “Polyamine Transporter Selective Compounds as Anti-Cancer Agents.” Otto Phanstiel, U.S. Pat. No. 8,497,398 (Jul. 30, 2013); “Polyamine Transport Selective Therapeutic Agents with Enhanced Stability.” Otto Phanstiel IV and Aaron Muth, World Patent, WO 2013148230 A1, Oct. 3, 2013; “Fluorescent Cytotoxic Compounds Specific for the Cellular Polyamine Transport System”, Otto Phanstiel IV, U.S. Patent 2013-0337494-A1 (Dec. 19, 2013); “Polyamine Transporter Selective Compounds as Anticancer agents”, Otto Phanstiel, U.S. Patent US-2014-0057989-A1 (Feb. 27, 2014); “Polyamine Transport Inhibitors as Novel Therapeutics”, US-2012-0172449-A1, Pub Date: Jul. 5, 2012; see also, U.S. patent application Ser. No. 14/388,856, U.S. Patent Application No. 62/097,896, U.S. patent application Ser. No. 12/113,540, U.S. patent application Ser. No. 13/953,667, U.S. patent application Ser. No. 14/862,907, U.S. patent application Ser. No. 13/379,191, U.S. patent application Ser. No. 12/113,540, U.S. patent application Ser. No. 12/754,962, U.S. patent application Ser. No. 13/835,708, and US Patent Publication No. 2012/01725499A1, all of which are herein incorporated by reference to the extent they relate to polyamine transport inhibitors and polyamine biosynthesis inhibitors.

### REFERENCES

1. Casero, R. A., Jr.; Marton, L. J., Targeting polyamine metabolism and function in cancer and other hyperproliferative diseases. Nature Reviews. Drug Discovery 2007, 6 (5), 373-90.
2. Watanabe, S.; Kusama-Eguchi, K.; Kobayashi, H.; Igarashi, K., Estimation of polyamine binding to macromolecules and ATP in bovine lymphocytes and rat liver. J Biol Chem 1991, 266 (31), 20803-20809.
3. Poulin, R.; Casero, R. A.; Soulet, D., Recent advances in the molecular biology of metazoan polyamine transport. Amino Acids 2012, 42 (2-3), 711-23.
4. Abdulhussein, A. A.; Wallace, H. M., Polyamines and membrane transporters. Amino Acids 2013, in press, PMID:23851697; doi: 10.1007/s00726-013-1553-6.
5. Aouida, M. P., R.; Ramotar, D., The Human Carnitine Transporter SLC22A16 Mediates High Affinity Uptake of the Anticancer Polyamine Analogue Bleomycin-A5. J. Biol. Chem. 2010, 285 (9), 6275-6284.
6. Muth, A.; Kamel, J.; Kaur, N.; Shicora, A. C.; Ayene, I. S.; Gilmour, S. K.; Phanstiel, O., IV, Development of Polyamine Transport Ligands with Improved Metabolic Stability and Selectivity against Specific Human Cancers. J Med Chem 2013, 56, 5819-5828.
7. Wang, C.; Delcros, J.-G.; Biggerstaff, J.; Phanstiel, O., Synthesis and biological evaluation of N1-(anthracen-9-ylmethyl)triamines as molecular recognition elements for the polyamine transporter. J. Med. Chem. 2003, 46, 2663-2671;
8. Samal, K.; Zhao, P.; Kendzicky, A.; Yco, L. P.; McClung, H.; Gerner, E.; Burns, M.; Bachmann, A. S.; Sholler, G., AMXT-1501, a novel polyamine transport inhibitor, synergizes with DFMO in inhibiting neuroblastoma cell proliferation by targeting both ornithine decarboxylase and polyamine transport. International Journal of Cancer 2013, 133 (6), 1323-1333.
9. Weeks, R. S., Vanderwerf, S. M.; Carlson, C. L.; Burns, M. R.; O'Day, C. L.; Cai, F.; Devens, B. H.; Webb, H. K., Novel lysine-spermine conjugate inhibits polyamine transport and inhibits cell growth when given with DFMO. Exp. Cell Res. 2000, 261, 293-302.
10. Kaur, N.; Delcros, J.-G.; Imran, J.; Khaled, A.; Chehtane, M.; Tschammer, N.; Martin, B.; Phanstiel, O., IV, A comparison of chloroambucil- and xylene-containing polyamines leads to improved ligands for accessing the polyamine transport system. J. Med. Chem. 2008, 51, 1393-1401.
11. Soda, K., The mechanisms by which polyamines accelerate tumor spread. Journal of Experimental & Clinical Cancer Research 2011, 30, 95-104.
12. Kashiwagi, K.; Endo, H.; Kobayashi, H.; Takio, K.; Igarashi, K., Spermidine-preferential uptake system in Escherichia coli. ATP hydrolysis by PotA protein and its association with membrane. J Biol Chem 1995, 270 (43), 25377-25382.
13. Kashiwagi, K.; Kuraishi, A.; Tomitori, H.; Igarashi, A.; Nishimura, K.; Shirahata, A.; Igarashi, K., Identification of the putrescine recognition site on polyamine transport protein PotE. J Biol Chem 2000, 275 (46), 36007-36012.
14. Kashiwagi, K.; Miyamoto, S.; Nukui, E.; Kobayashi, H.; Igarashi, K., Functions of potA and potD proteins in spermidine-preferential uptake system in Escherichia coli. J Biol Chem 1993, 268 (26), 19358-19363.
15. Tomitori, H.; Kashiwagi, K.; Sakata, K.; Kakinuma, Y.; Igarashi, K., Identification of a gene for a polyamine transport protein in yeast. J Biol Chem 1999, 274 (6), 3265-3267.
16. Heinick, A.; Urban, K.; Roth, S.; Spies, D.; Nunes, F.; Phanstiel, O., IV; Liebau, E.; Luersen, K., Caenorhabditis elegans P5B-type ATPase CATP-5 operates in polyamine transport and is crucial for norspermidine-mediated suppression of RNA interference. FASEB J. 2010, 24 (1), 206-217.
17. Pinto, F.; Corradi, G. R.; Hera, D. P.; Adamo, H. P., CHO cells expressing the human P(5)-ATPase ATP13A2 are more sensitive to the toxic effects of herbicide paraquat. Neurochemistry International 2012, 60 (3), 243-248).
18. Grillo, M. A.; Colombatto, S., Polyamine transport in cells. Biochemical Society Transactions 1994, 22 (4), 894-898.
19. Seiler, N.; Delcros, J. G.; Moulinoux, J. P., Polyamine transport in mammalian cells. An update. The International Journal of Biochemistry & Cell Biology 1996, 28 (8), 843-861.
20. Byers, T. L.; Kameji, R.; Rannels, D. E.; Pegg, A. E., Multiple pathways for uptake of paraquat, methylglyoxal bis(guanylhydrazone), and polyamines. The American Journal of Physiology 1987, 252 (6 Pt 1), C663-C669.
21. De Smedt, H.; Van den Bosch, L.; Geuns, J.; Borghgraef, R., Polyamine transport systems in the LLC-PK1 renal epithelial established cell line. Biochimica et Biophysica Acta 1989, 1012 (2), 171-177.
22. Kumagai, J.; Jain, R.; Johnson, L. R., Characteristics of spermidine uptake by isolated rat enterocytes. The American Journal of Physiology 1989, 256 (5), G905-G910.
23. Ruchko, M.; Gillespie, M. N.; Weeks, R. S.; Olson, J. W.; Babal, P., Putrescine transport in hypoxic rat main PASMCs is required for p38 MAP kinase activation. American Journal of Physiology. Lung cellular and molecular physiology 2003, 284 (1), L179-L186.
24. Seiler, N.; Dezeure, F., Polyamine transport in mammalian cells Int. J. Biochem. 1990, 22, 211-218.
25. Mitchell, J. L.; Simkus, C. L.; Thane, T. K.; Tokarz, P.; Bonar, M. M.; Frydman, B.; Valasinas, A. L.; Reddy, V. K.; Marton, L. J., Antizyme induction mediates feedback limitation of the incorporation of specific polyamine analogues in tissue culture. Biochem. J. 2004, 384 (Pt 2), 271-279.
26. Meyskens Jr, F. L. G., E. W., Development of Difluoromethylornithine (DFMO) as a chemoprevention agent. Clin. Cancer Res. 1999, 5, 945-951.
27. Burns, M. R., Carlson, C. L.; Vanderwerf, S. M.; Ziemer, J. R.; Weeks, R. S.; Cai, F.; Webb, H. K.; Graminski, G. F., Amino acid/spermine conjugates: Polyamine amides as potent spermidine uptake inhibitors. J. Med. Chem. 2001, 44, 3632-3644.
28. Burns, M. R.; Graminski, G. F.; Weeks, R. S.; Chen, Y.; O'Brien, T. G., Lipophilic lysine-spermine conjugates are potent polyamine transport inhibitors for use in combination with a polyamine biosynthesis inhibitor. J. Med. Chem. 2009, 52, 1983-1993.
29. Phanstiel, O. IV; Archer, J. J., Design of polyamine transport inhibitors as therapeutics. In Polyamine Drug Discovery, Casero, R. A. W., P. M., Ed. Royal Society of Chemistry: 2012; pp 162-190.
30. Soulet, D. G., B.; Rivest, S.; Audette, M.; Poulin, R., A fluorescent probe of polyamine transport accumulates into intracellular acidic vesicles via a two-step mechanism. J. Biol. Chem. 2004, 279, 49355-49366.
31. Belting, M.; Persson, S.; Fransson, L.-A., Proteoglycan involvement in polyamine uptake. Biochem. J. 1999, 338, 317-323.
32. Belting, M.; Mani, K.; Jonsson, M.; Cheng, F.; Sandgren, S.; Jonsson, S.; Ding, K.; Delcros, J-G.; Fransson, L-A., Glypican-1 is a vehicle for polyamine uptake in mammalian cells: A pivotal role for nirosothiol-derived nitric oxide. J. Biol. Chem. 2003, 278, 47181-47189.
33. Welch, J. E.; Bengston, P.; Svensson, K.; Wittrup, A.; Jenniskens, G. J.; Ten Dam, G. B.; Van Kuppevelt, T. H.; Belting, M., Single chain fragment anti-heparan sulfate antibody targets the polyamine transport system and attenuates polyamine-dependent cell proliferation. Int. J. Oncol. 2008, 32, 749-756.
34. (Basu Roy, U. K.; Rial, N. S.; Kachel, K. L.; Gerner, E. W., Activated K-RAS increases polyamine uptake in human colon cancer cells through modulation of caveolar endocytosis. Mol. Carcinogen. 2008, 47, 538-553.
35.; Uemura, T.; Stringer, D. E.; Blohm-Mangone, K. A.; Gerner, E. W., Polyamine transport is mediated by both endocytic and solute carrier transport mechanisms in the gastrointestinal tract. American Journal of Physiology. Gastrointestinal and Liver Physiology 2010, 299 (2), G517-G522).

### ADDITIONAL REFERENCES

1. Design of Polyamine Transport Inhibitors as Therapeutics. Otto Phanstiel IV; Jennifer J. Archer, in Polyamine Drug Discovery, P. Woster and R. A. Casero, eds., RSC Publishing, 2012. 162-187. ISBN 9781849731904. Gives an overview of known PTIs all of which are based upon polyamine scaffolds or polycationic scaffolds which mimic the native polyamines.
2. Polyamine transport inhibitors: Design, Synthesis and Combination therapies with Difluoromethylornithine. Aaron Muth, Jennifer Archer, Nicolette Ocampo, Meenu Madan, Luis Rodriguez, and Otto Phanstiel IV. J. Med. Chem. 2014, 57, 348-363. Describes the synthesis and use of the trimer44NMe compound and its use as a PTI.
3. “Polyamine Transport Inhibitors as Novel Therapeutics”, US-2012-0172449-A1, Pub Date: Jul. 5, 2012. Covers the trisubstituted designs like trimer44NMe (6b) as PTI agents

It should be borne in mind that all patents, patent applications, patent publications, technical publications, scientific publications, and other references referenced herein are hereby incorporated by reference in this application in order to more fully describe the state of the art to which the present invention pertains. The teachings of all patents and other references cited herein are incorporated herein by reference in their entirety to the extent they are not inconsistent with the teachings herein.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise.

While a number of embodiments of the present invention have been shown and described herein in the present context, such embodiments are provided by way of example only, and not of limitation. Numerous variations, changes and substitutions will occur to those of skill in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

What is claimed is:

1. A method for detecting cancer cells susceptible to treatment with a polyamine transport inhibitor (PTI), comprising:
receiving a cell sample from a patient;
presenting a first reagent, a second reagent, a third reagent, and a fourth reagent to the cell sample to detect differential levels of ATP13A3, c-myc, Cav-1, and Cav-2 or nucleic acid sequences encoding the same, in the cell sample, wherein the first reagent is specific to ATP13A3, the second reagent is specific to c-myc, the third reagent is specific to Cav-1, and the fourth reagent is specific to Cav-2 in the cell sample, under conditions to detect binding of the first, second, third and fourth reagents to ATP13A3, c-myc, Cav-1, and Cav-2, respectively, or hybridization of the first, second, third and fourth reagents to the nucleic acid sequences,
determining that cancer cells in the patient are treatable with a polyamine transport inhibitor if a higher level of ATP13A3, Cav-2, and c-myc and a basal or low level of Cav-1, or nucleic acid sequences encoding same, than in normal cells was determined to be present in the cell sample.

2. The method of claim 1 wherein first, second, third and fourth reagents are antibodies.

3. The method of claim 2, wherein conditions to detect binding of the first, second, third and fourth reagents comprises conducting an enzyme linked immunosorbent assay or a western blot method, wherein the first, second, third and fourth reagents comprise a detectable marker.

4. The method of claim 1, wherein the first reagent, second reagent, third reagent, and fourth reagent are primers directed to sequences that encode ATP13A3, c-myc, Cav-1, and Cav-2 respectively, and wherein conditions to detect hybridization comprises subjecting the cell sample and first, second, third and fourth reagents to PCR.

5. The method of claim 1, wherein the cell sample comprises intact cells or tissue, and the first reagent comprises a first antibody_{A} to detect ATP13A3, the second reagent comprises a first antibody_{B} to detect c-myc, the third reagent comprises a first antibody_{C} to detect Cav-1, and the fourth reagent comprises a first antibody_{D} to detect Cav-2 in the cell sample, and wherein conditions to detect binding comprises conducting an immunocytochemistry or immunohistochemistry method on the cell sample.

6. The method of claim 5, wherein the first antibodies each comprise a label.

7. The method of claim 5, further comprising presenting a second antibody_{A} specific to and capable of binding the first antibody_{A}, a second antibody_{B} specific to and capable of binding the first antibody_{B}, a second antibody_{C} specific to and capable of binding the first antibody_{C}, and a second antibody_{D} specific to and capable of binding the first antibody_{D}.

8. The method of claim 7, wherein each of the second antibodies comprise a label.

9. (canceled)

10. (canceled)

11. (canceled)

12. The method of claim 1, wherein the method further comprises identifying high levels of expression of c-Raf in the cancer cells.

13. The method of claim 1, further comprising administering a therapeutically effective amount of a polyamine biosynthesis inhibitor and/or a PTI to the patient upon determining cancer cells in the patient are treatable.

14-69. (canceled)

70. An assay system to identify cells having polyamine transport activity (PTA) for targeted treatment of cells, said assay system comprising:
a substrate configured to bind proteins in a cell sample; and
two or more antibodies selected from the group consisting of a primary antibody_{A} that detects ATP13A3, a primary antibody_{B} that detects c-myc, a primary antibody_{C} that detects Cav-1 and a primary antibody_{D} that detects Cav-2 in a sample, wherein upon contacting the cell sample and the primary antibodies_{A-D} to the substrate, the primary antibodies_{A-D} bind specifically to proteins ATP13A3, c-myc, Cav-1, and Cav-2, respectively, in the cell sample, wherein the primary antibodies_{A-D} are each individually linked to different colored fluorophores, such that visualization comprises a colored panel readout of the relative expression of each protein and differential expression levels of the proteins can be detected,
and wherein a detection of high levels of ATP13A3, c-myc, and/or Cav-2, and/or a low level of Cav-1 in the sample is indicative of PTA (polyamine transport activity) in the cell sample.

71. (canceled)

72. (canceled)

73. A method for detecting cancer cells susceptible to treatment with a polyamine transport inhibitor (PTI), comprising:
receiving a cell sample from a patient;
presenting at least a first and a second reagent to the cell sample to detect differential levels of two or more of ATP13A3, c-myc, Cav-1, and Cav-2 or nucleic acid sequences encoding the same, in the cell sample, under conditions to detect binding of the at least first and second reagents to two or more of ATP13A3, c-myc, Cav-1, and Cav-2, or hybridization of at least the first and second reagents to nucleic acid sequences.

74. The method of claim 73, wherein the first and second reagents are specific to ATP13A3 and Cav-1, respectively, and wherein the method further comprises determining that cancer cells in the patient are treatable with a polyamine transport inhibitor if a higher level of ATP13A3 and a lower or basal level of Cav-1, or nucleic acid sequences encoding same, relative to normal cells, was determined to be present in the cell sample; and wherein the hi her level of ATP13A3 is at least 50-100% higher than basal level, and wherein the lower level of Cav-1 is within 20% of basal level, relative to normal cells.

75. (canceled)

76. The method of claim 73, wherein the first and second reagents are specific to ATP13A3 and c-myc, respectively, and wherein the method further comprises determining that cancer cells in the patient are treatable with a polyamine transport inhibitor if a higher level of ATP13A3 and a higher level of c-myc, or nucleic acid sequences encoding same, relative to normal cells was determined to be present in the cell sample; and wherein the higher level of ATP13A3 is at least 50-100% higher than basal level, and wherein higher level of c-myc is at least 50-100% higher than basal level, relative to normal cells.

77. (canceled)

78. The method of claim 73, wherein the first and second reagents are specific to Cav-1 and Cav-2, respectively, and wherein the method further comprises determining that cancer cells in the patient are treatable with a polyamine transport inhibitor if a higher level of Cav-2 and a lower or basal level of Cav-1, or nucleic acid sequences encoding same, relative to normal cells was determined to be present in the cell sample.

79. The method of claim 73, wherein the first and second reagents are specific to ATP13A3 and Cav-2, respectively, and wherein the method further comprises determining that cancer cells in the patient are treatable with a polyamine transport inhibitor if a higher level of ATP13A3 and a higher level of Cav-2, or nucleic acid sequences encoding same, relative to normal cells was determined to be present in the cell sample; and wherein the higher level of ATP13A3 is at least 50-110% higher than basal level and wherein higher level of Cav-2 is within 20% of basal level relative to normal cells.

80. (canceled)

81. The method of claim 73, wherein the first and second reagents are specific to c-myc and Cav-1, respectively, and wherein the method further comprises determining that cancer cells in the patient are treatable with a polyamine transport inhibitor if a higher level of c-myc and a lower or basal level of Cav-1, or nucleic acid sequences encoding same, relative to normal cells was determined to be present in the cell sample, and wherein the higher level of c-myc is at least 50-100% higher than basal level and wherein the lower level of Cav-1 is within 20% of basal level.

82. (canceled)

83. The method of claim 73, wherein the first and second reagents are specific to c-myc and Cav-2, respectively, and wherein the method further comprises determining that cancer cells in the patient are treatable with a polyamine transport inhibitor if a higher level of c-myc and a higher level of Cav-2, or nucleic acid sequences encoding same, relative to normal cells was determined to be present in the cell sample; and wherein the higher level of c-myc is at least 50-100% higher than basal level, and wherein higher level of Cav-2 is at least 50-100% higher than basal level.

84. (canceled)

85. (canceled)

86. (canceled)

87. (canceled)

88. (canceled)

89. (canceled)

90. The method of claim 73, wherein the first and second reagents are primary antibodies selected from a group consisting of a primary antibody_{A} that detects ATP13A3, a primary antibody_{B} that detects c-myc, a primary antibody_{C} that detects Cav-1, and a primary antibody_{D} that detects Cav-2

91. The method of claim 90, wherein the primary antibody_{A} is HPA-029471.


SEQUENCE LISTING (4 sequences)

SEQ ID NO: 1 - protein, 439 aa, Homo sapiens
MPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 2 - protein, 178 aa, Homo sapiens
MSGGKYVDSEGHLYTVPIREQGNIYKPNNKAMADELSEKQVYDAHTKEIDLVNRDPKHLNDDVVKIDFEDVIAEPEGTHSFDGIWKASFTTFTVTKYWFYRLLSALFGIPMALIWGIYFAILSFLHIWAVVPCIKSFLIEIQCISRVYSIYVHTVCDPLFEAVGKIFSNVRINLQKEI

SEQ ID NO: 3 - protein, 1226 aa, Homo sapiens
MDREERKTINQGQEDEMEIYGYNLSRWKLAIVSLGVICSGGFLLLLLYWMPEWRVKATCVRAAIKDCEVVLLRTTDEFKMWFCAKIRVLSLETYPVSSPKSMSNKLSNGHAVCLIENPTEENRHRISKYSQTESQQIRYFTHHSVKYFWNDTIHNFDFLKGLDEGVSCTSIYEKHSAGLTKGMHAYRKLLYGVNEIAVKVPSVFKLLIKEVLNPFYIFQLFSVILWSTDEYYYYALAIVVMSIVSIVSSLYSIRKQYVMLHDMVATHSTVRVSVCRVNEEIEEIFSTDLVPGDVMVIPLNGTIMPCDAVLINGTCIVNESMLTGESVPVTKTNLPNPSVDVKGIGDELYNPETHKRHTLFCGTTVIQTRFYTGELVKAIVVRTGFSTSKGQLVRSILYPKPTDFKLYRDAYLFLLCLVAVAGIGFIYTIINSILNEVQVGVIIIESLDIITITVPPALPAAMTAGIVYAQRRLKKIGIFCISPQRINICGQLNLVCFDKTGTLTEDGLDLWGIQRVENARFLSPEENVCNEMLVKSQFVACMATCHSLTKIEGVLSGDPLDLKMFEAIGWILEEATEEETALHNRIMPTVVRPPKQLLPESTPAGNQEMELFELPATYEIGIVRQFPFSSALQRMSVVARVLGDRKMDAYMKGAPEAIAGLCKPETVPVDFQNVLEDFTKQGFRVIALAHRKLESKLTWHKVQNISRDAIENNMDFMGLIIMQNKLKQETPAVLEDLHKANIRTVMVTGDSMLTAVSVARDCGMILPQDKVIIAEALPPKDGKVAKINWHYADSLTQCSHPSAIDPEAIPVKLVHDSLEDLQMTRYHFAMNGKSFSVILEHFQDLVPKLMLHGTVFARMAPDQKTQLIEALQNVDYFVGMCGDGANDCGALKRAHGGISLSELEASVASPFTSKTPSISCVPNLIREGRAALITSFCVFKFMALYSIIQYFSVTLLYSILSNLGDFQFLFIDLAIILVVVFTMSLNPAWKELVAQRPPSGLISGALLFSVLSQIIICIGFQSLGFFWVKQQPWYEVWHPKSDACNTTGSGFWNSSHVDNETELDEHNIQNYENTTVFFISSFQYLIVAIAFSKGKPFRQPCYKNYFFVFSVIFLYIFILFIMLYPVASVDQVLQIVCVPYQWRVTMLIIVLVNAFVSITVEESVDRWGKCCLPWALGCRKKTPKAKYMYLAQELLVDPEWPPKPQTTTEAKALVKENGSCQIITIT

SEQ ID NO: 4 - protein, 162 aa, Homo sapiens
MGLETEKADVQLFMDDDSYSHHSGLEYADPEKFADSDQDRDPHRLNSHLKLGFEDVIAEPVTTHSFDKVWICSHALFEISKYVMYKFLTVFLAIPLAFIAGILFATLSCLHIWILMPFVKTCLMVLPSVQTIWKSVTDVIIAPLCTSVGRCFSSVSLQLSQD